(19) United States
(12) Patent Application Publication
(10) Pub. No.: US 2025/0054623 A1
(43) Pub. Date: Feb. 13, 2025

(54) ARTIFICIAL INTELLIGENCE MODELING FOR MULTI-LINGUISTIC DIAGNOSTIC AND SCREENING OF MEDICAL DISORDERS

(71) Applicant: The Regents of the University of California, Oakland, CA (US)

(72) Inventors: Peter Yellowlees, Davis, CA (US); Michelle Burke PARISH, Davis, CA (US); Steven Richard Chan, Davis, CA (US)

(73) Assignee: The Regents of the University of California, Oakland, CA (US)

(21) Appl. No.: 18/573,925

(22) PCT Filed: Jun. 24, 2022
(86) PCT No.: PCT/US2022/035019

§ 371 Date: Dec. 22, 2023

Related U.S. Application Data
(60) Provisional application No. 63/214,733, filed on Jun. 24, 2021.

(51) Int. Cl. G16H 50/20; G06F 40/58; G10L 15/02; G10L 15/26; G16H 10/60

(52) U.S. Cl. CPC G16H 50/20; G06F 40/58; G10L 15/02; G10L 15/26; G16H 10/60

(57) ABSTRACT

Disclosed herein are methods and systems for a training a model for real-time patient diagnosis. A system may include a computer configured to receive audio data and video data of a clinical encounter, the audio data comprising spoken words by an entity and the video data depicting the entity; retrieve clinical data regarding the entity; execute a model using the words of the audio data and the retrieved clinical data regarding the entity as input, the execution causing the model to output a plurality of clinical diagnoses for the entity; concurrently render the corresponding video data and audio data and the plurality of clinical diagnoses via a computing device associated with a user; and store an indication of a selected clinical diagnosis of the plurality of clinical diagnoses responsive to receiving a selection of the clinical diagnosis at the computing device.

### CROSS-REFERENCE TO RELATED APPLICATION

This application is a national stage application under 35 U.S.C. § 371 that claims the benefit of and priority to P.C.T. Patent Application No. PCT/US2022/035019, filed Jun. 24, 2022; which claims priority to U.S. Provisional Application No. 63/214,733, filed Jun. 24, 2021, the entirety of each of which is incorporated by reference herein.

### STATEMENT REGARDING FEDERALLY SPONSORED RESEARCH OR DEVELOPMENT

This invention was made with Government support under grant nos. R01-HS021477 and R01-HS024949, each awarded by the Agency for Healthcare Research and Quality (AHRQ). The Government has certain rights in this invention.

### TECHNICAL FIELD

This application relates generally to using artificial intelligence modeling to predict and optimize screening and treatment for mental health and other medical disorders

### BACKGROUND

The need for better self-directed screening and treatment for mental health conditions is a significant problem in health care. For example, depression, a very common and potentially treatable psychiatric illness, is so often missed or ignored that it is the second most expensive illness globally in terms of productive days lost. In the USA over 50% of behavioral health patients are treated in primary care settings, 67% of people with behavioral health disorders do not get behavioral health treatment, that 80% of behavioral health patients will visit a primary care provider (PCP) annually while 67% of PCPs report being unable to access outpatient behavioral healthcare for their patients. This lack of care occurs despite the theoretical availability of multiple validated screening questionnaires for mental illnesses like depression. Key drawbacks to these tools are they are not user friendly and are not often integrated into electronic health records (EHR). The result is that screening measures available now are not broadly used despite governmental financial support, and are not set up for repeat measuring and monitoring.

The mental health care system has been significantly affected by the COVID-19 pandemic, with what has been described as a follow-on mental health pandemic. Both the World Health Organization and the Centers for Disease Control have published reports describing greater community levels of depression, anxiety, substance use, domestic violence, sexual abuse and related trauma, and likely suicides. Mental health professionals have been required to develop new telepsychiatry protocols and digital systems to help their patients who are staying at home, while the number of consultations nationwide has dramatically escalated.

Diagnostic screening for depression and many other psychiatric disorders is currently methodologically basic, primarily depending on simple validated questionnaires. Provider initiated screening tools are underutilized and depression is commonly missed in the primary care setting and in particularly vulnerable populations such as individuals with limited English proficiency or limited access to healthcare. This problem has been exacerbated by COVID-19, which has been correlated with an uptick in psychiatric disorders and has further limited access to patient treatment.

Telepsychiatry, in the form of videoconferencing, is an important tool in behavioral health care. Synchronous Telepsychiatry (STP), where consultations are done in real-time and are interactive, has increased access to care making psychiatric experts available in areas with provider shortages. Research has demonstrated high rates of patient satisfaction and similar clinical outcomes to traditional in-person care for many disorders, including depression and anxiety. Telemedicine utilization across all disciplines had already been anticipated to grow exponentially to a 430 billion dollar industry by 2025, before the use of telepsychiatry dramatically increased during the COVID-19 pandemic. During the COVID-19 pandemic telepsychiatry became a core healthcare tool for most psychiatrists in the United States. Many clinics rapidly converted to telepsychiatry, with a number describing the experience and the changes required including the move to in-home consultations, or virtual house calls. For example, the large University of California Davis (UCD) behavioral health outpatient clinic, saw a successful conversion from approximately 97% in-person consultations, to 100% virtual consultations in 3 days. A survey conducted by the American Psychiatric Association during the COVID-19 pandemic found that by June of 2020 85% of 500 surveyed American psychiatrists were using telepsychiatry with more than 75% of their patients, compared with about 3% prior to COVID-19.

National telehealth statistics derived from 60 contributing private insurers to the Fair Health database showed an increase of 2,816% in telehealth consultations in all disciplines in December 2020 compared with December 2019. Telehealth consultations comprised 6.5% of all consultations nationally in their database in that year, with 47% of the patients being seen for primarily mental health reasons. The National Center for Health Statistics' reported a total of 883 million outpatient consultations nationally in 2018. Projecting from the insurance statistics about 3% of these in 2020 were telepsychiatry visits (by video or phone), an approximate total of 26 million such visits.

Despite such success, with STP being the current standard telepsychiatry practice, administrative and technical challenges exist, especially around scheduling of telepsychiatrists and patients. STP itself is simply a virtual extension of in-person care which cannot be scaled to enable one provider to see more patients, for multiple providers/experts to easily review a single patient encounter for multiple opinions across disciplines, or to include additional patient information/data streams to improve the accuracy of depression and other mental health assessment tools.

### SUMMARY

For the aforementioned reasons, there is a need to increase access to health screening, diagnosis, and repeat monitoring through asynchronous telepsychiatry (ATP) and/or AI assisted screening, diagnosis, and treatment. Care utilizing ATP and AI assisted screening and diagnosis is particularly important in addressing this mental health crisis because this tool allows for an automated end-to-end system that can adapt a computer model (e.g., an artificial intelligence model) to automatically simulate a patient's diagnosis and treatment plan and to optimize the patient's diagnosis and treatment plan in a manner that does not depend on a healthcare provider's subjective skills and understanding. Additionally, there is a need to increase access to medical care to vulnerable populations, such as those without direct access to medical care (such as the unhoused) and those without direct access to medical care in their primary language (such as Spanish speaking populations.) These factors may particularly effect minority populations and other vulnerable groups. Additionally, ATP can provide an innovative solution to treat people in their homes as part of the COVID-19 pandemic response and the ATP collaborative care model leverages the expertise of psychiatrists so that they can oversee the treatment of larger numbers of patients

In one aspect, the present disclosure is directed to a method for asynchronous telemedicine. In some implementations, the method comprises receiving, by a processor of a computing device, a first set of words having a first attribute; and predicting, by the processor, a second set of words having a second attribute. In some embodiments, the method further comprises executing an artificial intelligence model to identify a patient characteristic, the artificial intelligence model trained using a training dataset comprising data associated with a plurality of previously assigned characteristics on a plurality of previous patients.

### BRIEF DESCRIPTION OF THE DRAWINGS

Non-limiting embodiments of the present disclosure are described by way of example with reference to the accompanying figures, which are schematic and are not intended to be drawn to scale. Unless indicated as representing the background art, the figures represent aspects of the disclosure.

FIG. 1 illustrates a flow diagram of a process executed in a multi-linguistic diagnostic and screening analysis system, according to an embodiment.

FIG. 2A illustrates a flow diagram of a process executed in a multi-linguistic diagnostic and screening analysis system, according to an embodiment.

FIG. 2B illustrates components multi-linguistic diagnostic and screening analysis system, according to an embodiment.

FIG. 3 illustrates a flow diagram of a process executed in a multi-linguistic diagnostic and screening analysis system, according to an embodiment.

FIG. 4 illustrates components of a multi-linguistic diagnostic and screening analysis system, according to an embodiment.

FIG. 5 illustrates a flow diagram of a process for training a model for real-time patient diagnosis, according to an embodiment.

FIGS. 6-17 illustrate graphical user interfaces generated and displayed in a multi-linguistic diagnostic and screening analysis system, according to an embodiment.

FIGS. 18-25 illustrate graphical user interfaces generated and displayed in a multi-linguistic diagnostic and screening analysis system, according to an embodiment.

FIG. 26 illustrates the results of an asynchronous multi-linguistic diagnostic and screening analysis system, according to one embodiment.

FIGS. 27-32 illustrate graphical user interfaces generated and displayed in a multi-linguistic diagnostic and screening analysis system, according to an embodiment.

### DETAILED DESCRIPTION

Reference will now be made to the illustrative embodiments depicted in the drawings, and specific language will be used here to describe the same. It will nevertheless be understood that no limitation of the scope of the claims or this disclosure is thereby intended. Alterations and further modifications of the inventive features illustrated herein, and additional applications of the principles of the subject matter illustrated herein, which would occur to one skilled in the relevant art and having possession of this disclosure, are to be considered within the scope of the subject matter disclosed herein. Other embodiments may be used and/or other changes may be made without departing from the spirit or scope of the present disclosure. The illustrative embodiments described in the detailed description are not meant to be limiting of the subject matter presented.

Diagnostic screening for depression and other mental health disorders is currently methodologically basic, primarily depending on simple validated questionnaires. Provider initiated screening tools are underutilized and depression is commonly missed in the primary care setting and in particularly vulnerable populations such as individuals with limited English proficiency.

By including data and a series of other patient collected data streams, it is possible to build an improved depression or other mental health assessment tool to screen for depression or other disorders with a combination of patient reported data and physiological markers of depression. An Asynchronous Telepsychiatry App (ATPApp) can allow uploading of audio and video files (together or separately) of diagnostic interviews with patients in any language. These interviews may be transcribed in the upload language, and translated into a new language (for example translating a Spanish spoken interview to English). Although referred to generally as an interview, in various implementations, the audio and/or video files may record a patient-provider dialogue, a patient self-assessment questionnaire, a computerized or scripted interview without a provider present, a dynamic scripted interview via a machine learning-based decision tree, or a free-form monologue or “open” questionnaire with no provider present. These interviews may be combined with additional electronic health records and/or passive data streams such as from apps or mobile devices. Additional data collected simultaneously with audio and video may include any type and form of physiological or physical data (e.g. heart rate, heart rate variability, skin surface temperature, nystagmus, blood pressure, breathing rate, gesture frequency or size, pose, etc.), and may be provided for review in synchronization with the accompanying audio and/or video. The system may allow a psychiatrist, healthcare provider, or mental health expert to review the original audio and video in the language they were recorded in, with subheadings in a different language if required or with a text-to-speech translation, and then record comments and diagnoses that they derive from observing the interview, in some cases in concurrent with a review of the additional electronic health records. The system may also allow experts from multiple fields to review one data source and provide their opinion. This may allow review by more than one discipline (e.g. psychiatry and pulmonary medicine) for co-occurring or complex conditions (e.g., depression and post-COVID pulmonary syndrome) to improve coordination of care

In an embodiment, the ATPApp may be a self-assessment screening tool, for instance for depression. This may include a patient-facing interface for ATPApp that may allow patients to audio and video self-record as they are automatically interviewed via a decision tree series of questions, including some validated diagnostic questionnaires, and clinically relevant history questions. In some embodiments, this interface may replace an interview conducted by a trained provider. This may allow patients to be easily screened via an app on their devices.

In some embodiments, language transcription and translation engines may be integrated into the application to allow for multilingual interviews to be conducted. In some embodiments, voice, facial and movement recognition engines may be integrated. In some embodiments as discussed above, the system may additionally record a variety of other external physiological measures of vital signs, heart rate variability, skin conductance and additional passive data. This additional data may be analyzed manually or with a physiological analysis engine for purposes such as allowing screening assessments to be more diagnostically accurate, determining treatment plans, and detecting comorbidities.

Artificial intelligence and machine learning algorithms trained on previously recorded patients may be used to increase the diagnostic accuracy of the continuing recordings. These may be used to calculate a diagnostic screening risk stratification level and/or determine the need to send the enhanced video for further analysis by an expert clinician or multiple specialists to evaluate complex issues (e.g. a psychiatrist and a neurologist may both consult on the same patient with a complex condition or multiple conditions). AI models can be trained based on historical data and/or trained using granular data (e.g., based on a specific patient), such that the AI model's predictions are specific to a particular patient. Using the methods and systems described herein, a server (e.g., a central server or a computer associated with a specific clinic) may diagnose and treat mental health issues using specially trained AI models.

In some embodiments, treatment may be asynchronous where an interview is performed and data is collected and sent to a provider who provides diagnosis and/or treatment at a later time. In other embodiments, treatment may be synchronous where the interview is performed and data is collected concurrently with diagnosis and/or treatment by a human or artificial intelligence (AI) system.

By implementing the systems and methods described herein, a provider may avoid the costs and processing resources that are typically required to diagnose and treat mental health issue. Moreover, the solution may expand access to diagnosis and treatment to vulnerable and at-risk populations, allow treatment in multiple languages, find correlating variables to positive outcomes, and allow for cross-checking diagnosis and treatment between AI models and providers.

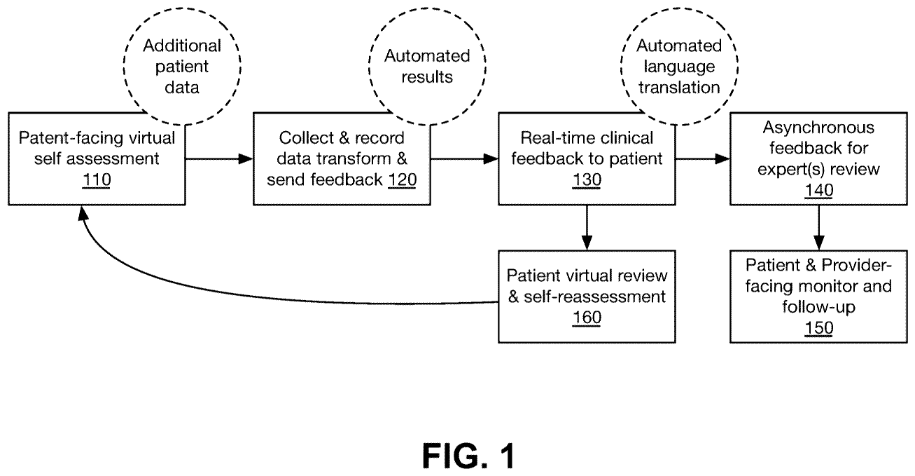

FIG. 1 illustrates a flow diagram of a process executed in a multi-linguistic diagnostic and screening analysis system, according to an embodiment.

At step 110, a patient may engage in a virtual self-assessment. The patient may engage with a digital health interview with a pre-programmed decision tree audio questionnaire. In some embodiments, the patient may engage with in a digital health interview with a chat bot, or a synchronously generated series of question from an artificial intelligence engine generated trained on data from previous sessions. The selected corpus used to train the engine may be recorded human sessions between a patient and a practitioner. The selected corpus may be previous sessions recording from the application. The selected corpus may be previous sessions recorded from the same respondent. A more general corpus may be filtered or selected based on a series of patient characteristics or traits input into the system. The AI system may alter the phrasing of questions to determine a correlation between question phrasing, patient background, and a positive outcome. The artificial intelligence system may be rewarded for questions or interviews that result in a positive outcome. The positive outcome may be things such as a correlation with a correct diagnosis, a minimization of system resources, an optimized treatment plan, expert (human) positive feedback, or an improvement in patient health.

The digital health interview may be held in the patient's native language or requested language. In some cases, the requested language may be directly input into the system, such as by a button or keypad entry. In other cases, the language may be detected based on a spoken or written set of words from the patient. In some cases, the system displays subtitles in the patient's native or requested language at the bottom of the video screen during the interview.

In some instances, the patient may engage with a human interviewer. The interviewer may be a non-expert interviewer or group of interviewers. The interviewer may speak the interview questions in the patient's native or requested language. Alternately, the interviewer may speak the interview questions in the interviewer's native language and the questions may be translated into the patient's native or requested language in either written or auditory form. Similarly, the patient's answers may be translated to the interviewer's native language. Examples of this would include written translations on an iPad or other screen or spoken translations in an earpiece. The system may use translation protocols generated or already on the market. The artificial intelligence system may track and analyze if the method of translation, written or verbal form, or other characteristics of the translation correlate with positive outcomes or other notable related variables.

The interviewer may be given a set of questions. The set of questions may be generated by artificial intelligence, such as by the methods outlined above. The interviewer may be given a decision tree based on the response of the patient during the interview. The interviewer may have elected to conduct the interview on the basis of a set of criteria. For instance, a social worker or nonprofit worker may utilize a set of criteria to determine that an interview would be appropriate. Alternately, the patient may request that an interview be conducted.

At step 110, a video of the interview may be recorded. The video may record only the patient or the video may record the patient as well as others taking part in the interview. The type and position of the video recorder may be analyzed using an artificial intelligence system to determine if there is a correlation to positive or negative outcomes. The video may be analyzed using an artificial intelligence system to determine if other characteristics such as the identity of the interviewer, the time of day, the number of interviewers, characteristics related to the setting of the interview (such as the presence of plants or color tone), the position of the interviewer relative to the patient, the types and position of chairs correlate to positive outcomes. The video may be analyzed to determine interactions between the interviewer and the patient. Interactions may include body language, distance between the patient and the interview, and the tone of the patient and interviewer, among others.

In some embodiments, additional information may be collected during the interview. This additional information may include such things as facial characteristics and movement, body language and movement, and tone. Language characteristics, such as word choice and language may be collected. In some embodiments, physiological data such as vital signs, heart rate variability, skin conductance and similar data may be collected. In some embodiments, the multi-linguistic diagnostic and screening analysis system may be trained to alter the questions, tone, or other aspects of the interview in real time based on the physiological data collected.

For instance, an artificial intelligence or machine learning model may be trained on previous data to detect helpful interventions due to a shift in tone or body language of the patient. The system may train on previously captured comparative data to determine cues of factors that correlate with interview questions, tone, and other factors resulting in a positive outcome. The multi-linguistic diagnostic and screening analysis system may analyze the video, language, movement and physiological data captured in real time to increase the accuracy and value of the interview using the asynchronous nature of the internet and previously captured comparative data.

In some embodiments, the multi-linguistic diagnostic and screening analysis system may give real-time improvement to the interview. For instance, the multi-linguistic diagnostic and screening analysis system may translate the interview between the preferred language of the interviewer (or default language of the digital interviewer) and the preferred language of the patient. This translation may take the form of written words, for instance, in the form of subtitles or a translation on a pad or screen, or auditory words, for example, a spoken translation in an earpiece.

In some embodiments, an audio recording will be taken instead of a video recording. The artificial intelligence model may suggest a format to be used for the interview based on characteristics of the patient or suspected diagnosis.

At step 120, the video or audio file may be added to the patient's file along with any other patient characteristics, electronic medical record, or similar clinical information. The clinical information may be used to determine comorbidities and/or to help form a diagnosis. Additionally, the clinical information may be used in determining a treatment plan.

At step 130, the multi-linguistic diagnostic and screening analysis system may analyze the input data in real time. This data may be supplemented with clinical information from step 120. The analysis system may use this information to alter the interview questions and parameters in real time. The analysis system may calculate a risk stratification of a diagnosis, for example, high, medium, or low for any psychiatric or medical diagnosis. The analysis system may also calculate a confidence interval or level of certainty for the diagnosis. If the level of certainty is above a threshold, the analysis system may relay to the patient the risk stratification for the diagnosis. In some cases, the analysis system may relay to the patient a definitive diagnosis. In some embodiments, the analysis system may feed back to the patient a treatment plan or next step. In some embodiments, the analysis system may feed back to the patient a referral to the relevant care provider for the suspected diagnosis. If the level of certainty is below a threshold, the analysis system may feed back additional questions or testing. Alternately, the analysis system may feed back to the patient a timeline for diagnosis.

At step 140, some or all the data collected at step 120 may be send to an expert for further review. This data may include some or all of the following: the interview recording, the interview transcript, additional medical records, the risk stratification calculated by the analysis system, relevant factors used by the analysis system to determine the risk stratification (factors or variables that impacted the analysis systems score), diagnostic tests, among others. The interview may be modified to the preferred language of the expert, for instance using subtitles or audio “dubbing”. The interview may be modified to remove portions of the interview the analysis system determines is not relevant to the diagnosis. For instance, the analysis system may select certain interview frames or video segments where questions were asked to show the provider first—for instance, the video segments showing questions and answers that led to an increase in the certainty threshold of the analytical system's diagnosis. The interview may be modified to include relevant additional data at points during the interview.

The expert reviewer may agree or disagree with the diagnosis, risk stratification, and certainty threshold determined by the analysis system. The analysis system may use this feedback to train and revise the model used for the determination, for instance, using feedback to train an optimization algorithm. The expert reviewer may agree or disagree with the factors, variables, and frames used by the system to determine the diagnosis, risk stratification, and certainty threshold. These factors may be removed or down weighted by the algorithm. In some instances, if the expert reviewer and the analysis system disagree on a diagnosis, and the analysis system diagnosis is above a certainty threshold, the data collected at step 120 may be sent to a second expert reviewer for confirmation.

At step 150, the diagnostic information may be used for individual treatment or for population health management. In some embodiments, the provider and patient may meet synchronously either face to face or over video or audio to relay the diagnosis and/or treatment plan. In some embodiments, the diagnosis and/or treatment plan may be relayed digitally in written, audio or video form. In some embodiments, the diagnosis and/or treatment plan may be relayed to a third party, such as a social worker or counselor, to relay to the patient one-on-one. A follow-up plan may also schedule a set of meetings or interactions.

At step 160, the patient may select to complete a virtual review of the diagnosis and/or treatment plan as well as submit feedback regarding the process. The patient may decide to complete a virtual self-reassessment at the time of the diagnosis or later after treatment. Part of the suggested treatment plan may include the patient completing a virtual review and self-reassessment at treatment intervals.

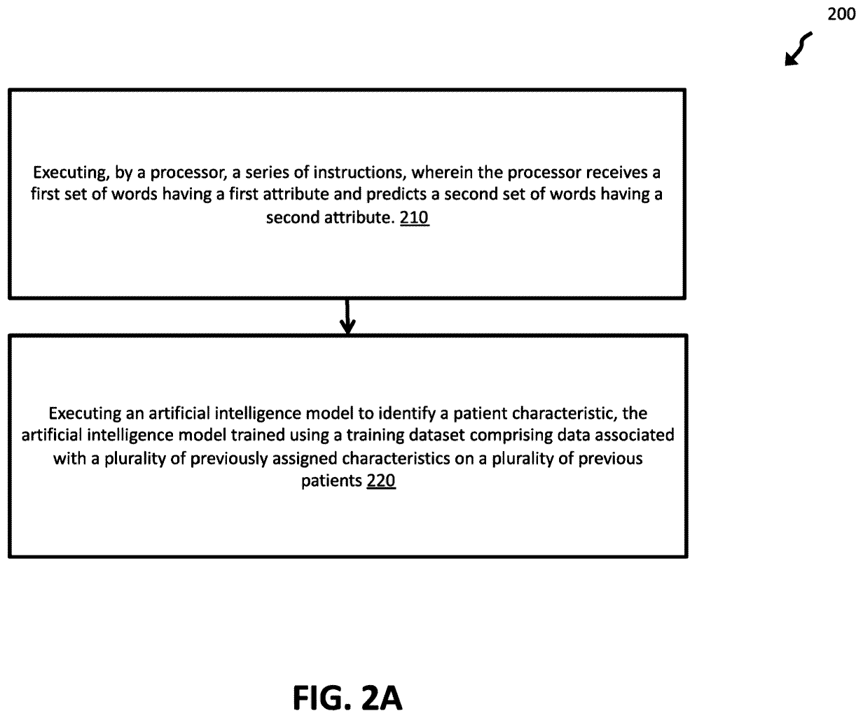

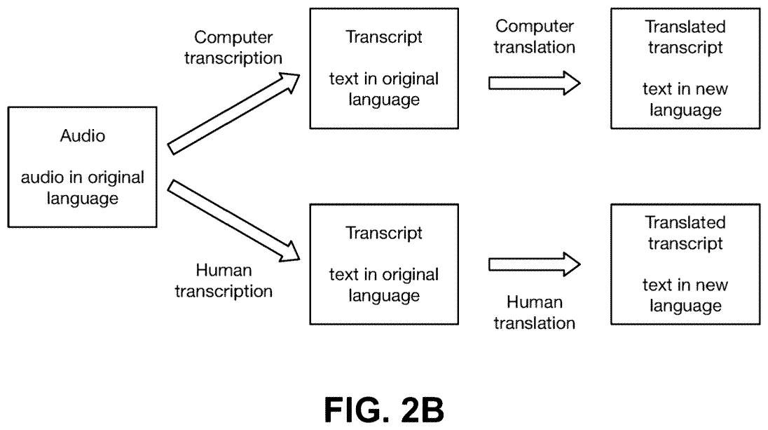

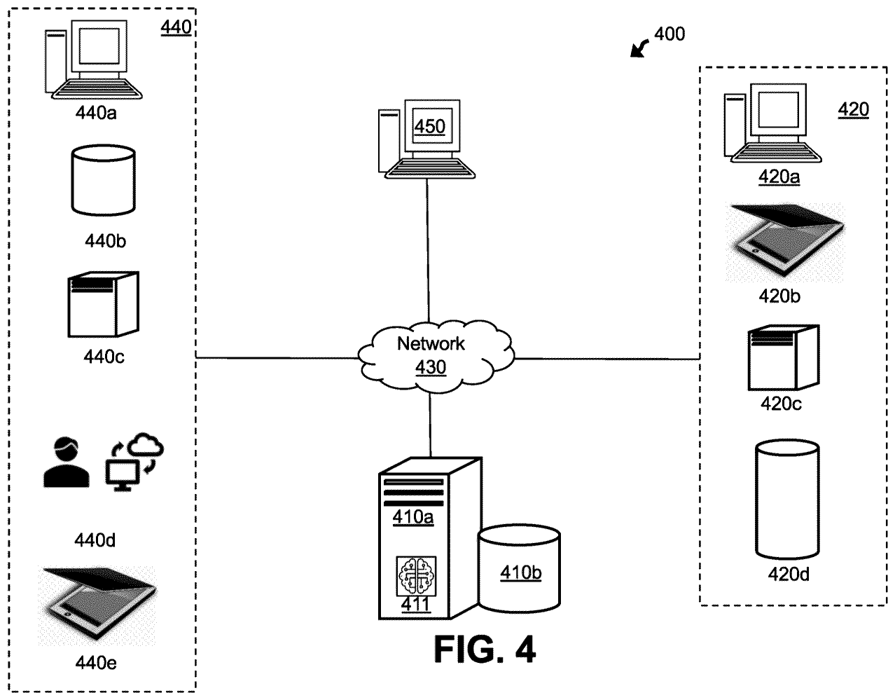

FIG. 2A illustrates a flow diagram of a process executed in a multi-linguistic diagnostic and screening analysis system, according to an embodiment. The method 200 includes steps 210-220. However, other embodiments may include additional or alternative steps, or may omit one or more steps altogether. The method 200 is described as being executed by an analytics server, such as the analytics server 410a described in FIG. 4). However, one or more steps of method 200 may be executed by any number of computing devices operating in the distributed computing system described in FIG. 4. For instance, one or more computing devices may locally perform part or all of the steps described in FIG. 2A or a cloud device may perform such steps.

At step 210, if needed, the analytics server may execute an artificial intelligence model to translate questions from the default language or the language of the interviewer to the language of the patient. If needed, the analytics server may execute an artificial intelligence model to translate the responses from the language of the patient to the language of the interviewer or the default system of the patient. For instance, the analytics server may execute, by a processor, a series of instructions, wherein the processor receives a first set of words having a first attribute and predicts a second set of words having a second attribute.

At step 220, the analytics server may execute an artificial intelligence model to identify a diagnosis, the artificial intelligence model trained using a training dataset comprising data associated with a plurality of previously generated diagnoses on a plurality of previous patient. For instance, the training dataset may include previous patients and all collected data and recordings. The training dataset may include a predicted diagnosis and any updates or revisions to the diagnosis.

The analytics server may access an AI model (e.g., neural network, convolutional neural network, or any other machine-learning model such as random forest or a support vector machine) trained based on a training dataset corresponding to previously treated patients. The analytics server may apply a patient's information (e.g., comorbidities of the patent, physical attributes of the patient, history) to the trained AI model. As a result, the trained AI model may predict a diagnosis for the patient.

Before accessing or executing the AI model, the analytics server may train the AI model using data associated with previously diagnosed and/or treated patients to predict a diagnosis and/or treatment plan for a patient. The AI model may be trained by the analytics server or by an external data processing system. Previously diagnosed and/or treated patients, as used herein, may correspond to patients who were treated by a particular clinic or a set of clinics or by the analysis system. The analytics server may generate a training dataset that includes data associated with previously treated patients and their diagnosis and/or treatment plans (e.g., relevant answers, plan objectives, additional medical data, or any other data associated with how the diagnosis was reached or treatment was implemented). Additionally or alternatively, the analytics server may augment the training dataset using patient data associated with other clinics.

The analytics server may include various attributes associated with a previously treated patient, such as the patient's physical attributes (e.g., height and weight) and health attributes (e.g., comorbidities) in the training dataset. The analytics server may also collect treatment data associated with the patient's treatments. An example of treatment data associated with previously treated patients may include medication, behavior modification, or retest frequency. Another example of data associated with a patient's treatment may include clinical goals that correspond to the patient's treatment. The clinical goals may be used in conjunction with the patient plan such that the training dataset includes a holistic view of each patient's treatment. The analytics server may use the clinical goals to determine what treatment plan was used based on the diagnosis and clinical goals for the patient.

Using this information, the analytics server may train the AI model. Using the diagnosis based on the data and treatment and in light of the plan objectives and clinical goals, the analytics server may use various training techniques to train the AI model. For supervised training methods, the analytics server may use labeling information, provided by a clinical expert, to train the AI model. The analytics server may also account for an individual's corresponding clinical goals and plan objectives. This additional information may provide additional context around the treatment plan. For instance, two different patients may have received treatment. However, each patient may have a different clinical goal and plan objectives or medical history. Therefore, the analytics server may train the AI model using contextual data around each patient.

The analytics server may identify hidden patterns that are unrecognizable using conventional methods (e.g., manual methods or computer-based methods). The analytics server may then augment this recognition with analyzing various other attributes, such as patient attributes and/or clinical goals and plan objectives.

The analytics server may also include any diagnosis of the patient who was previously treated within the training dataset. For instance, the analytics server may retrieve diagnoses produced before, during, or after the patient's treatment. The training dataset may also include treatment objectives (also referred to herein as the plan objective) associated with the previously treated patients. Treatment objective may refer to various predetermined rules and thresholds implemented by a provider or a clinician.

The training dataset may include diagnosis and treatment data associated with providers of different characteristics (e.g., geography, provider education training, type of provider such as psychiatrist, psychologist etc.) patients with different characteristics (e.g., that have different genders, weights, heights, body shapes, comorbidities, etc.), and/or that treat patients that have or have had different diseases (e.g., depression, bipolar, COVID-19, etc.). Consequently, the set of patients may include patients with a diverse set of characteristics that can be used to train the AI model to diagnose and treat a wide range of people.

The analytics server may generate the training dataset using various filtering protocols to control the training of the AI model. For instance, the training datasets may be filtered such that the training data set corresponds to previously treated patients at a particular provider and/or previously treated patients with a specific attribute (e.g., a disease type or a treatment modality). Additionally or alternatively, the analytics server may generate a training dataset that is specific to a particular patient. For instance, a treating provider may prescribe a series of therapy treatments for a particular patient. As the patient receives his/her therapy, the analytics server may collect data associated with each treatment and follow-up diagnosis. The analytics server may then generate a training dataset that is specific to the patient and includes data associated with that particular patient's treatments.

The analytics server may label the training dataset in such a way that the AI model can differentiate between desirable and undesirable outcomes. Labeling the training dataset may be performed automatically and/or using human intervention. In the case of automatically labeled training data, the analytics server may display various data attributes associated with a patient's diagnosis and/or treatment plan on an electronic platform where a medical expert can review the data and determine whether the diagnosis is acceptable. If the diagnosis and/or treatment plan is not acceptable, the model can be taught either by negative reinforcement of the diagnosis, or by drilling down on the data attributes used by the model. Using automatic and/or manual labeling, the analytics server may label the training dataset, such that when trained, the train AI model can distinguish between diagnoses.

After completing the training dataset, the analytics server may train the AI model using various machine-learning methodologies. The analytics server may train the AI model using supervised, semi-supervised, and/or unsupervised training or with a reinforcement learning approach. For example, the AI model may be trained to predict the dosage of medication needed, or the diagnosis of the patient. To do so, characteristic values of individual patients within the training dataset may be ingested by the AI model with labels indicating the correct predictions for the patients (e.g., examples of correct and incorrect diagnosis). The AI model may output diagnoses for individual patients based on their respective characteristics, and the outputs can be compared against the labels. Using back-propagation techniques, the AI model may update its weights and/or parameters based on differences between the expected output (e.g., the ground truth within the training dataset) and the actual outputs (e.g., outputs predicted by the AI model) to better predict future cases (e.g., new patients).

The analytics server may continue this training process until the AI model is sufficiently trained (e.g., accurate above a predetermined threshold). The computer may store the AI model in memory, in some cases upon determining the AI model has been sufficiently trained.

The AI model may be a multi-layered series of neural networks arranged in a hierarchical manner.

The AI model may ingest all the data within the training dataset to identify hidden patterns and connections between data points. To prevent the AI model from over-fitting, the analytics server may utilize various dropout regularization protocols. In an example, the dropout regulation may be represented by the following formula:

$$Dropoutrate = Ratemax \times ( \frac{Current Number of Filters}{Maximum number of filters} ) \qquad 1 / n$$

The choice for the dropout parameters may be iteratively calculated using empirical data, until the gap between the validation loss and training loss does not tend to increase during training. To assess the overall performance of the AI model, the analytics server may select a set of patients (e.g., test set). The analytics server may then perform a cross validation procedure on the remaining patients. The analytics server may compare the predicted values with true and actual values within the training dataset (e.g., previous treatment of one or more patients). For instance, the analytics server may generate a value representing differences (actual vs. predicted) for the diagnosis and treatment for the test patient cases. Using this value, the analytics server may gauge how well the AI model is trained.

The analytics server may train the AI model such that the AI model is customized to predict values associated with the corresponding training dataset. For instance, if the analytics server trains an AI model using a training data set specific to a patient, the predicted result may be tailored for that patient. In another example, the analytics server may train the AI model, such that the AI model is trained for a specific type of disease (e.g., depression).

Upon completion of training, the AI model is ready to predict the diagnosis or treatment for patients. The analytics server may access the trained AI model via the cloud or by retrieving or receiving the AI model from a local data repository. For example, the analytics server may transmit a password or token to a device storing the AI model in the cloud to access the AI model. In another example, the analytics server may receive or retrieve the AI model either automatically responsive to the AI model being sufficiently trained or responsive to a GET request from the analytics server.

The analytics server may execute the trained AI model using a new set of data comprising characteristic values of patients receiving screening to generate a diagnosis. The analytics server may execute the AI model by sequentially feeding data associated with the patient. The analytics server (or the AI model itself) may generate a vector comprising values of the characteristics of the patient (e.g., height, weight, gender, occupation, age, history, body mass index, income, drug use, location, etc.) and input the vector into the AI model. The AI model may ingest the vector, analyze the underlying data, and output various predictions based on the weights and parameters the AI model has acquired during training.

The analytics server may receive values of characteristics of the patient and/or the diagnosis options from a user (e.g., a clinician, doctor, or the patient themselves) via a user interface and generate a feature vector that includes the values. Additionally or alternatively, the analytics server may retrieve values of characteristics of the patient from storage to include in the feature vector responsive to receiving an identifier of the patient. The analytics server may input the feature vector into the AI model and obtain an output from the AI model.

The analytics server may receive the characteristics for the patient based on a patient identifier that is provided via a user interface of the electronic platform. For example, a clinician may input the name of the patient into the user interface via an end-user device and the end-user device may transmit the name to the analytics server. The analytics server may use the patient's name to query a database that includes patient information and retrieve information about the patient such as the patient's electronic health data records. For instance, the analytics server may query the database for data associated with the patient's anatomy, such as physical data (e.g. height, weight, and/or body mass index), social data (e.g. poverty, food insecurity, loss), and/or other health-related data (e.g., blood pressure). The analytics server may also retrieve data associated with current and/or previous diagnoses or treatments received by the patient (e.g. data associated with the patient's previous mental health diagnosis or medical treatment).

If necessary, the analytics server may also analyze the patient's medical data records to identify the needed patient characteristics. For instance, the analytics server may query a database to identify the patient's body mass index (BMI). However, because many medical records are not digitized, the data processing system may not receive the patient's BMI value using simple query techniques. As a result, the analytics server may retrieve the patient's electronic health data and may execute one or more analytical protocols (e.g., natural language processing) to identify the patient's body mass index. The analytics server may also use these methods while preparing or pre-processing the training dataset.

The analytics server may receive additional data from one or more healthcare providers. For instance, a treating psychiatrist may access a platform generated/hosted by the analytics server and may add, remove, or revise data associated with a particular patient, such as patient attributes, mental health diagnoses, treatment plans, prescribed medication and the like.

The data received by the analytics server (e.g., patient/treatment data) may belong to three categories: numerical, categorical, and visual. Non-limiting examples of numerical values may include patient age, physical attributes, psychometric data, and other attributes that describe the patient. Non-limiting examples of categorical values may include severity or type of disease associated with the patient. Visual data may include body language, facial responses, mannerisms and the like.

The predicted value generated by the AI model may be used in various ways to further analyze, evaluate, and/or optimize the patient's diagnosis and/or treatment plan. In an example, the diagnosis predicted by the model may be displayed on a graphical user interface. In another example, the AI model's output may be ingested by another software application (e.g., plan optimizer). In yet another example, the AI model may be used to evaluate a treatment plan generated by another software solution (e.g., plan optimizer). Even though these examples are presented herein individually, the analytics server may perform any combination of above-described examples. For instance, the analytics server may predict a diagnosis for the patient and the AI model's predictions, and may transmit the predictions to another software solution to optimize the patient's treatment plan.

In addition to predicting the diagnosis discussed herein, the trained AI model may also predict a confidence score associated with the diagnosis and/or the treatment plan. The confidence score may correspond to a robustness value of the diagnosis predicted by the AI model.

In a non-limiting example, two diagnoses are analyzed by the AI model. The AI model indicates that both diagnosis plans comply with various rules and thresholds discussed herein (e.g., the diagnosis confidence interval is below a predetermined threshold). However, the AI model generates a confidence value that is significantly lower for the first diagnosis. This indicates that the first diagnosis is more likely to involve a comorbidity or incorrect diagnosis. The analytics server may also display an input field where the human reviewer can accept, deny, or revise the diagnosis and/or treatment plan.

Referring now to FIG. 2B, audio from the patient may be transcribed by a computer or human transcription. The transcription in the original language may then be translated using computer or human translation to obtain text in the preferred language of the system or the provider. Computer-aided transcription may take place using any one of the many systems which do or will exist such as Dragon, Dragon Medical, or a myriad of others. Computer-aided translation may take place using any of the many translation software that exists, for example, Google Translate, Bing Translator, Systran and many others. Alternately, transcription and translation may occur using the analytics server which is taught using a corpus of interviews in the original and target languages.

In some embodiments, a combination of systems is used for transcription and translation. For instance, transcription and translation may originally occur using off-the-shelf software. However, the transcriptions and translations may be intermittently checked by a human translator. The original audio input and translated data may form a corpus along with the corrections to teach the analytics server to correct the transcripted and/or translated word combinations it receives from the off-the-shelf software. The analytics server may be trained on a separate “corrective” corpus depending on the suspected diagnosis, characteristics of the patients, geographical location of the patients or other variables the analytics server determines affects the transcription and translation.

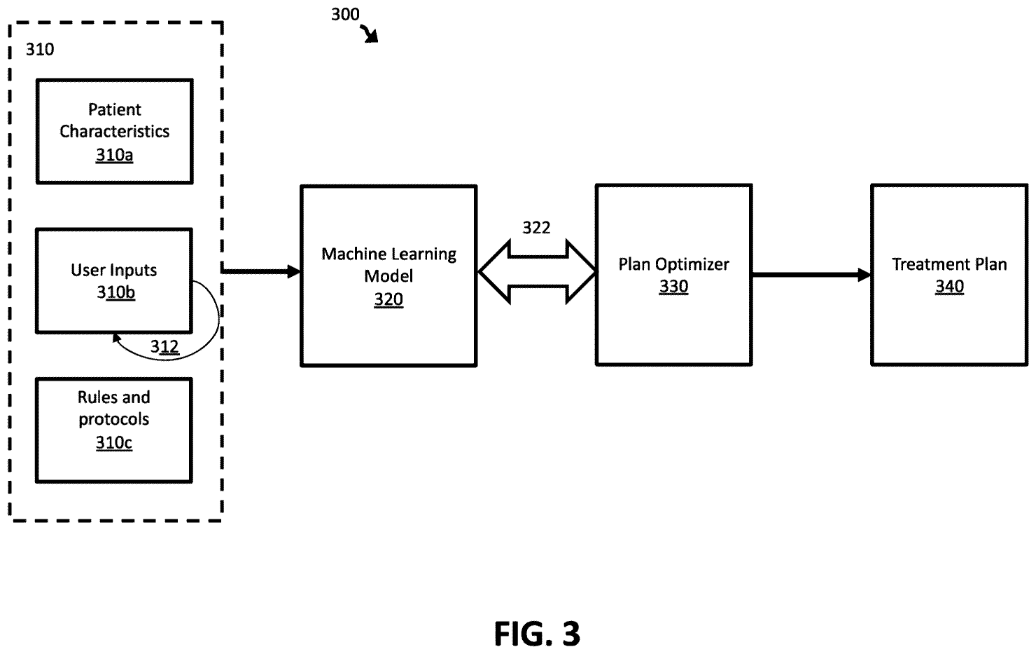

Referring now to FIG. 3, a non-limiting visual example of a workflow utilizing the methods and systems described herein is illustrated. In this non-limiting example 300, the analytics server provides prediction data to a plan optimizer 330 to generate a suggested treatment plan that is optimized for a patient. The analytics server may first collect patient data 310. The patient data may include patient anatomy data 310a, user inputs 310b (received via a user interface), and rules 310c for the patient's treatment (e.g., comorbidities or other plan objectives). The analytics server may train a machine-learning model 320 using previously diagnosed patients and treatment plans. The trained machine-learning model 320 may then identify various weights/parameters to predict a diagnosis and/or treatment plan for patients.

The analytics server may receive the patient's video, audio, or medical file and extract the needed patient data 310. The analytics server then executes the machine-learning model 320 using the patient data 310, such that the machine-learning model 320 ingests the patient data 310 and predicts a diagnosis and treatment plan. For instance, the machine-learning model 320 may determine a predicted diagnosis based upon the interview and medical history of the patient. As described above, the machine-learning model 320 is trained using previously performed treatments and their corresponding patient, user inputs, and other data associated with the patient's treatment (e.g., clinic rules or special instructions received from the treating provider).

In some embodiments, the results generated via the machine-learning model 320 may be ingested by the plan optimizer 330. The plan optimizer 330 may be a treatment planning and/or monitoring software solution. The plan optimizer 330 may analyze various factors associated with the patient and the patient's treatment to generate and optimize a treatment plan for the patient (e.g., medication, behavior modification, therapy). The plan optimizer 330 may utilize various cost function analysis protocols where the diagnosis is evaluated in light of the other factors, such as comorbidities. When the plan optimizer completes the patient's treatment plan, the plan optimizer 330 may transmit the suggested treatment plan 340 to one or more electronic devices where a user (e.g., clinician) can review the suggested plan. For instance, the suggested treatment plan 340 may be displayed on a computer of a clinic where a psychiatrist can review the treatment plan.

In addition to the embodiments described above, the analytics server may use the trained AI model to independently generate a treatment plan or to independently evaluate a plan generated by the plan optimizer. The analytics server may retrieve a treatment plan for a patient comprising a medication or other treatment plan associated with the patient. The analytics server may communicate with a software solution configured to generate a treatment plan for a patient, such as the plan optimizer discussed herein. The plan optimizer may execute various analytical protocols to identify and optimize a patient's treatment plan. For instance, the plan optimizer may retrieve patient diagnosis, patient data (e.g., physical data, disease data, and the like). The plan optimizer may also retrieve plan objectives associated with the patient's treatment. The plan optimizer may use various analytical protocols and cost functions to generate a treatment plan for the patient using the patient data. Using the above-mentioned data, the plan optimizer may generate a treatment plan for the patient that includes various treatment parameters, such as suggested medication, behavioral changes, or therapy.

The analytics server may then retrieve the suggested treatment from the plan optimizer. The analytics server may execute the AI model to evaluate the plan, as generated by the plan optimizer. Alternately, the treatment plan may be generated by the AI model directly, or the treatment plan may be generated by a human clinician.

The analytics server may execute the trained AI model using previous patient data and results and may compare the diagnosis and/or treatment plan to the treatment plans either (1) frequently used for a similar patient or (2) the diagnosis and/or treatment plan which has historically led to the most favorable outcome in the training data. The analytics server 410a may transmit an alert if the diagnosis and/or treatment plan does not match that suggested by the analytics server. In some cases, the analytics server may only transmit the alert if the confidence is above a specified threshold. The notification may alert the healthcare providers involved with the patient's diagnosis and/or treatment does not match the suggested diagnosis and/or treatment plan. The healthcare provider may review the anomalies predicted by the AI model to accept or reject the diagnosis and/or treatment plan.

In addition to training the AI model as discussed above, the analytics server may use user interactions to further train and re-calibrate the AI model. When an end user performs an activity on the electronic platform that displays the results predicted via the AI model, the analytics server may track and record details of the user's activity. For instance, when a predicted result is displayed on a user's electronic device, the analytics server may monitor the user's electronic device to identify whether the user has interacted with the predicted results by editing, deleting, accepting, or revising the results. The analytics server may also identify a timestamp of each interaction, such that the analytics server records the frequency of modification and/or duration of revision/correction.

The analytics server may utilize an application-programming interface (API) to monitor the user's activities. The analytics server may use an executable file to monitor the user's electronic device. The analytics server may also monitor the electronic platform displayed on an electronic device via a browser extension executing on the electronic device. The analytics server may monitor multiple electronic devices and various applications executing on the electronic devices. The analytics server may communicate with various electronic devices and monitor the communications between the electronic devices and the various servers executing applications on the electronic devices.

Using the systems and methods described herein, the analytics server can have a formalized approach to generate, optimize, and/or evaluate a diagnosis or treatment plan or dose distribution in a single automated framework based on various variables, parameters, and settings that depend on the patient and/or the patient's treatment. The systems and methods described herein enable a server or a processor associated with (e.g., located in) a clinic to generate a diagnosis or treatment plan that is optimized for individual patients, replacing the need to depend on a clinician's subjective skills and understanding.

As will be described below, a server (referred to herein as the analytics server) can train an AI model (e.g., neural network or other machine-learning models) using historical treatment data and/or patient data from the patient's previous treatments. In a non-limiting example, the analytics server may transfer, or a processor of a clinic may otherwise access, the trained AI model to a processor associated with the clinic for calibration and/or evaluation of treatment plans. FIG. 4 is an example of components of a system in which the analytics server operates. Various other system architectures that may include more or fewer features may utilize the methods described herein to achieve the results and outputs described herein. Therefore, the system depicted in FIG. 4 is a non-limiting example.

FIG. 4 illustrates components of a multi-linguistic diagnostic and screening analysis system 400. The system 400 may include an analytics server 410a, system database 410b, AI models 411, electronic data sources 420a-d (collectively electronic data sources 420), end-user devices 440a-c (collectively end-user devices 440), and an administrator computing device 450. Various features depicted in FIG. 4 may belong to a provider at which patients may receive mental health or other medical treatment,

The above-mentioned components may be connected to each other through a network 430. Examples of the network 430 may include, but are not limited to, private or public LAN, WLAN, MAN, WAN, and the Internet. The network 430 may include wired and/or wireless communications according to one or more standards and/or via one or more transport mediums. The communication over the network 430 may be performed in accordance with various communication protocols such as Transmission Control Protocol and Internet Protocol (TCP/IP), User Datagram Protocol (UDP), and IEEE communication protocols. In one example, the network 430 may include wireless communications according to Bluetooth specification sets or another standard or proprietary wireless communication protocol. In another example, the network 430 may also include communications over a cellular network, including, for example, a GSM (Global System for Mobile Communications), CDMA (Code Division Multiple Access), or EDGE (Enhanced Data for Global Evolution) network.

The system 400 is not confined to the components described herein and may include additional or other components, not shown for brevity, which are to be considered within the scope of the embodiments described herein.

The analytics server 410a may generate and display an electronic platform configured to use various computer models 411 (including artificial intelligence and/or machine-learning models) to optimize the diagnosis and treatment of mental health disorders or treatment plans

The electronic platform may include graphical user interfaces (GUIs) displayed on each electronic data source 420, the end-user devices 440, and/or the administrator computing device 450. An example of the electronic platform generated and hosted by the analytics server 410a may be a web-based application or a website configured to be displayed on different electronic devices, such as mobile devices, tablets, personal computer, and the like. In a non-limiting example, a provider operating the provider device 420b may access the platform, input patient attributes or characteristics and other data, and further instruct the analytics server 410a to optimize the patient's diagnosis. The analytics server 410a may utilize the methods and systems described herein to optimize diagnosis and display the results on one of end-user devices 440. The analytics server 410a may display the predicted diagnosis on the provider device 420b itself as well.

The analytics server 410a may host a website accessible to users operating any of the electronic devices described herein (e.g., end users), where the content presented via the various webpages may be controlled based upon each particular user's role or viewing permissions. The analytics server 410a may be any computing device comprising a processor and non-transitory machine-readable storage capable of executing the various tasks and processes described herein. Non-limiting examples of such computing devices may include workstation computers, laptop computers, server computers, and the like. While the system 400 includes a single analytics server 410a, the analytics server 410a may include any number of computing devices operating in a distributed computing environment, such as a cloud environment.

The analytics server 410a may execute software applications configured to display the electronic platform (e.g., host a website), which may generate and serve various webpages to each electronic data source 420 and/or end-user devices 440. Different users may use the website to view and/or interact with the predicted results.

The analytics server 410a may be configured to require user authentication based upon a set of user authorization credentials (e.g., username, password, biometrics, cryptographic certificate, and the like). The analytics server 410a may access the system database 410b configured to store user credentials, which the analytics server 410a may be configured to reference in order to determine whether a set of entered credentials (purportedly authenticating the user) match an appropriate set of credentials that identify and authenticate the user.

The analytics server 410a may also store data associated with each user operating one or more electronic data sources 420 and/or end-user devices 440. The analytics server 410a may use the data to weigh interactions while training various AI models 411 accordingly. For instance, the analytics server 410a may indicate that a user is a healthcare provider whose inputs may be monitored and used to train the machine-learning or other computer models 411 described herein.

The analytics server 410a may generate a user interface (e.g., host or present a webpage) that presents information based upon a particular user's role within the system 400. In such implementations, the user's role may be defined by data fields and input fields in user records stored in the system database 410b. The analytics server 410a may authenticate the user and may identify the user's role by executing an access directory protocol (e.g. LDAP). The analytics server 410a may generate webpage content that is customized according to the user's role defined by the user record in the system database 410b.

The analytics server 410a may receive RTTP data (e.g., patient and treatment data for previously implemented treatments) from a user (healthcare provider) or retrieve such data from a data repository, analyze the data, and display the results on the electronic platform. For instance, in a non-limiting example, the analytics server 410a may query and retrieve medical images from the database 420d and combine the medical images with treatment data received from a provider operating the provider device 420b. The analytics server 410a may then execute various models 411 (stored within the analytics server 410a or the system database 410b) to analyze the retrieved data. The analytics server 410a then displays the results via the electronic platform on the administrator computing device 450, the electronic healthcare provider device 420b, and/or the end-user devices 440.

The electronic data sources 420 may represent various electronic data sources that contain, retrieve, and/or input data associated with patients and their treatment (e.g., patient data, diagnosis, and treatment plans). For instance, the analytics server 410a may use the clinic computer 420a, provider device 420b, server 420c (associated with a provider and/or clinic), and database 420d (associated with the provider and/or the clinic) to retrieve/receive data associated with a particular patient's treatment plan.

End-user devices 440 may be any computing device comprising a processor and a non-transitory machine-readable storage medium capable of performing the various tasks and processes described herein. Non-limiting examples of an end-user device 440 may be a workstation computer, laptop computer, tablet computer, and server computer. In operation, various users may use end-user devices 440 to access the GUI operationally managed by the analytics server 410a. Specifically, the end-user devices 440 may include clinic computer 440a, clinic database 440b, clinic server 440c, a medical device and the like.

The administrator computing device 450 may represent a computing device operated by a system administrator. The administrator computing device 450 may be configured to display data retrieved by the analytics server 410a (e.g., various analytic metrics and/or field geometry) where the system administrator can monitor various models 411 utilized by the analytics server 410a, electronic data sources 420, and/or end-user devices 440; review feedback; and/or facilitate training or calibration of the AI model 411 that are maintained by the analytic server 410a.

The analytics server 410a may store AI models 411 (e.g., neural networks, random forest, support vector machines, etc.) The analytics server 410a may train the AI models 411 using patient data, diagnosis, and/or treatment data associated with patients who were previously treated. For instance, the analytics server 410a may receive patient data (e.g., physical attributes and diagnosis) and diagnosis (e.g., data corresponding the mental health diagnosis of the patient) from any of the data sources 420.

The analytics server 410a may then generate one or more sets of labeled (or sometimes unlabeled) training dataset indicating the patient diagnosis and/or treatment plan (and whether they are acceptable or not). The analytics server 410a may input the set of labeled training dataset into the stored AI models 411 for training (e.g., supervised, unsupervised, and/or semi-supervised) to train the AI models 411 to predict the mental health diagnosis for future screening. The analytics server 410a may continue to feed the training data into the AI models 411 until the AI models 411 are accurate to a desired threshold and store the AI models 411 in a database, such as the database 410b. In the illustration of FIG. 4, AI models 411 are shown as being executed by the analytics server 410a, but may be stored on analytics server 410a or system database 410b.

The AI models stored in the database 410b may correspond to individual types of screened disorders, different types of provider groups, types of patients, location of screening geographical regions, genders, or other variables found to correlate with commonalities. For example, each AI model 411 may be associated with an identifier indicating the provider, screened population, or a specific disease for which it is configured to diagnosis.

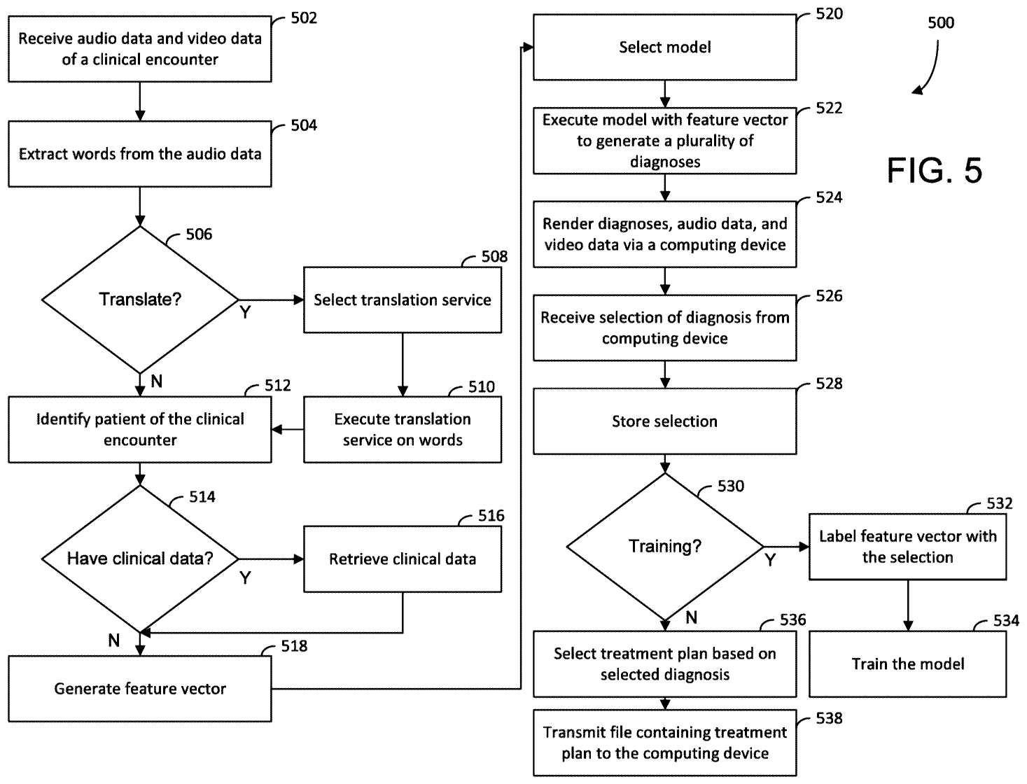

FIG. 5 is a flow chart of a method 500 for training a model for real-time patient diagnosis, according to some implementations. The method 500 may be performed by a data processing system (e.g., the analytics server 410a, shown and described with reference to FIG. 4). Performance of method 500 may enable the data processing system to train and use a series of models to diagnose patients from words spoken in a clinical encounter between a clinician or physician and an entity. In some embodiments, an entity is a patient. The method 500 may include any number of steps and the steps may be performed in any order.

At step 502, the data processing system may receive audio data and video data of a clinical encounter. The clinical encounter may be an instance of a patient speaking with a doctor or physician about a medical visit (e.g., psychotherapy visit, a visit at a medical clinic, or any other medical visit) discussing medical or other issues the patient may be experiencing. The audio data may be or include the sounds and audio of the conversation between the physician and the patient and any other sounds that are picked up by a microphone capturing the conversation. The video data may be a video or a collection of images of the patient talking to the physician during the clinical encounter.

The data processing system may receive the audio data and video data in a live data stream or as a file (e.g., a video file) containing the respective data. For example, in some embodiments, the data processing system may receive the audio data and video data in an audiovisual data stream during the clinical encounter and forward the audiovisual data stream to a client device for live playback. In some embodiments, the data processing system may receive the audio data and video data in a data file after the clinical encounter occurred. In such embodiments, the data processing system may execute the data file to process the audio data and video data from the data file as described herein.

At step 504, the data processing system may extract words from the audio data. The words may be natural language words (e.g., English, Spanish, French, or other language spoken to communicate between individuals in the world) that the patient and/or the physician speak over the course of the clinical encounter. The data processing system may extract the words, for example, by analyzing the sound waves (e.g., identifying the frequency of the soundwaves) in the audio data and identifying words (e.g., words from a database) that correspond to the different sound waves. In some instances, the data processing system may identify the individually spoken words using Fourier transform algorithms on the sound waves. The data processing system may identify both the speaker (e.g., the patient or the physician) and the individual words from the audio data using such methods. In some cases, upon identifying the words, the data processing system may label the words with the identified speaker by storing an indication of the speaker with the respective words in memory. The data processing system may extract and/or label each word the physician and patient speak to each other throughout the clinical encounter and store the extracted words and/or labels in memory.

At step 506, the data processing system may determine whether to translate the extracted words into another natural language. For instance, in some embodiments, the data processing system may be configured to translate extracted words from audio data from a first natural language into words of a second natural language (e.g., from English into Spanish). The data processing system may be configured in this manner by an administrator or by a user viewing the extracted words so the user can understand the words (e.g., the user may only speak Spanish, so the user may configure the data processing system to convert spoken words in English (or another language) into Spanish so the user can understand what is being said). In some cases, the data processing system may be configured not to translate the words into a second language.

If the data processing system is configured to translate the words into a second language, at step 508, the data processing system may select a translation service to use to perform the translation. A translation service may be a module or segment of code that is configured to translate text from one language to another language. A translation service may do so, for example, by matching words between the two languages and/or by using natural language processing techniques to translate or convert the words from one language to another. Such translation services may be configured to perform translations between any two languages.

The data processing system may select the translation service to use to perform the translation based on a determined accuracy of the available stored translation services (e.g., translation services stored in memory of the data processing system). For example, the data processing system may store multiple translation services that are each separately configured as different modules or segments of code. The data processing system may calculate the accuracy of each translation service by inserting the same set of words or text into each translation service and executing the code of the respective translation services. As a result, each translation service may output a translated version of the text. A reviewer may review and identify errors in the outputs of each translation service or the data processing system may compare the outputs to a “correct” version of the translated text. The reviewer and/or the data processing system may identify the number of errors in each version of translated text. The data processing system may calculate a correct percentage (e.g., number of words correct versus total possible number of words correct) or a total number of errors for each translation service based on output translated text. In some embodiments, the data processing system may calculate an error rate indicating a number of errors or a percentage of the translated text that contains an error. The data processing system may store indications of the percentage, total number of errors, or any such calculated value for each translation service in memory.

In some embodiments, the data processing system may select the translation service based on the calculated percentage or total number of errors. For example, in some embodiments, the data processing system may compare the total number of errors, the correct percentages, or the error rates of the translation services. Based on the comparison, the data processing system may identify the most accurate translation service as the translation service with the least number of errors, the translation service with the lowest error rate, or the translation service with the highest accuracy percentage. The data processing system may select the translation service based on the translation service having the least number of errors, the lowest error rate, or the highest accuracy percentage.

In some embodiments, the data processing system may select the translation service responsive to receiving a selection by a user. The data processing system may display strings identifying the translation services and the corresponding accuracy percentages or number of errors on a user interface to a user. The user may view the displayed data. The user may then select one of the translation services from the user interface. The data processing system may receive the selection and select the translation service to use for translations.

In some cases, the data processing system may use different translation services to translate text for different users. For example, the data processing system may present the same data on user interfaces to different users accessing different accounts. The different users may each select different translation services. Upon receiving the selections, the data processing system may store indications of the selections in the accounts. Accordingly, when performing a translation for a user accessing a particular user account, the data processing system may identify the identification of the translation service in the user account and select the translation service to use that corresponds to the translation.

At step 510, the data processing system may execute the selected translation service on the extracted words. The data processing system may do so, for example, by formatting the words into a readable string or vector that the translation service may use as input. The data processing system may then input the formatted words into the translation service and execute the translation service to generate translated text in the new language.

At step 512, the data processing system may identify the patient of the clinical encounter. The data processing system may identify the patient, for example, by using data stored in a database in memory or from a database stored on a third-party server. For instance, the data processing system may store a record of the clinical encounter in a database. The record may indicate various information about the patient, including the name of the patient, and/or the reason for the clinical encounter. The data processing system may identify the patient's name from the record as the patient that met with the physician of the clinical encounter. In another example, the data processing system may receive the name of the patient from the external device that transmitted data (e.g., audio and/or video data) for the clinical encounter to the data processing system.

At step 514, the data processing system may determine if there is any stored clinical data for the entity. The data processing system may do so by querying an internal database or by sending a request for data to another database. For example, the data processing system may query the internal database using the extracted patient's name as a search term. If the data processing system identifies a profile for the patient based on the query, the data processing system may search the profile (e.g., a data structure containing data about the patient) to determine if there is any clinical data (e.g., symptom data, medical history data, demographic data such as age, height, and gender, and/or other data related to a making a medical diagnosis).

At step 516, the data processing system may retrieve the clinical data from the profile. If the data processing system is not able to identify any clinical data from the profile, the data processing system may not retrieve any clinical data from the profile. In some embodiments, the data processing system may identify the name of the patient and/or clinical data about the patient from a user input the data processing system receives through a user interface.

In another example, the data processing system may transmit a request to an external computing device (e.g., another device that stores data about patients) for data about the patient. The request may include the name of the patient and a query for information about the patient. The external computing device may receive the request and search an internal database in memory of the external computing device. If the external computing device has any data about the patient, the external computing device may transmit the data to the data processing system. Otherwise, the external computing device may send an indication that the device does not have any clinical data stored for the patient.

At step 518, the data processing system may generate a feature vector from the extracted words and/or the clinical data. For example, if the data processing system retrieved clinical data about the patient, the data processing system may include values from or converted from the clinical in separate index values of the feature vector. The data processing system may additionally include words or values converted from words spoken by the patient and/or the physician (depending on the configuration of the data processing system) in separate index values of the feature vector. The words may be the words of the audio data pre- or post-translation. Accordingly, the data processing system may generate a feature vector for the clinical encounter that may be used by the data processing system to predict one or potential medical diagnoses for the patient.

In some embodiments, the data processing system may generate values from the words of the audio data using natural language processing techniques or machine learning techniques. For example, the data processing system may generate a text file from the spoken words and insert the text file into a machine learning model (e.g., a neural network) configured to generate an embedding (e.g., a vector) of a set number of numerical values from the text. In some embodiments or cases, the data processing system may similarly generate an embedding from the clinical data, if any, for the patient. The data processing system may concatenate the two embeddings to generate the feature vector. In some embodiments, the data processing system may concatenate the words and the clinical data together and generate an embedding from the concatenated data to use as a feature vector.

At step 520, the data processing system may select a model. The model may be a machine learning model (e.g., a neural network, a support vector machine, random forest, etc.) configured to predict medical diagnoses for different patients. The data processing system may select the model based on the model being trained to predict medical diagnoses for patients that have one or more identical characteristics to the patient (e.g., same gender, similar age range, similar height or weight range, similar symptoms, etc.). Such models may have been trained using data from patients that have the characteristics with which the model is associated. The data processing system may identify data about the patient from the clinical data and select the model to use to predict clinical diagnoses for the patient by comparing the identified data to metadata of the models (e.g., data associated with the models in memory). The data processing system may identify the model with metadata that matches the identified information or the model that has the highest amount of metadata that matches identified information and select the model to use to predict medical diagnoses for the patient.

At step 522, the data processing system may execute the selected model. The data processing system may execute the selected model using the generated feature vector as input. Upon execution, the model may apply its trained parameters and weights to the feature vector. In doing so, the model may generate confidence scores for a plurality of medical diagnoses for the patient.

At step 524, the data processing system may render (e.g., concurrently render) diagnoses, the audio data, and the video data via a computing device. The computing device may be a computing device that previously established a connection with the data processing system. The data processing system may render the audio data and the video data by transmitting the audio data and video data to the computing device. The computing device may play the audio data out of speakers of the computing device and render the video data displaying the video data as a video on a user interface on a display of the computing device. Accordingly, a user accessing or otherwise associated with the computing device may view the clinical encounter between the patient and the physician on the display.

In some embodiments, the data processing system may render words of the audio data on the user interface. The data processing system may render the originally extracted words from the audio data or translated words (or both) on the user interface, in some embodiments as an overlay to the video of the video data. The data processing system may transmit the words to the computing device with the audio data and the video data such that the words correspond (e.g., match) the words being spoken in the audio data and/or the video data (e.g., the words match the mouth movements of the physician and the patient). Accordingly, a hearing-impaired user or a user that does not speak the language being spoken in the audio data but that can read the transcribed words can understand the conversation between the physician and the patient.

In some embodiments, the data processing system also renders predicted diagnoses on the user interface being displayed on the computing device. To do so, the data processing system may select a defined number (e.g., five) of clinical diagnoses with the highest predicted confidence scores as calculated by the selected model and/or the clinical diagnoses with a confidence score that exceeds or otherwise satisfies a threshold. The data processing system may select the subset of clinical diagnoses based on any such criteria and transmit the subset of clinical diagnoses to the computing device with the audio data, video data, and/or words. The computing device may receive the subset of clinical diagnoses and display the clinical diagnoses on the user interface. In some embodiments, the data processing system may only select and transmit the clinical diagnosis with the highest confidence score to the computing device for display.

The client device may display the subset of clinical diagnoses on the user interface in a variety of manners. For example, the client device may display the subset of clinical diagnoses in ascending or descending order based on the confidence scores associated with the different clinical diagnoses. The client device may display the subset of clinical diagnoses concurrently with the other data of the clinical encounter. In some embodiments, the data processing system may display the clinical diagnoses with the corresponding confidence scores to illustrate to the user the likelihood that the different clinical diagnoses are correct.

At step 526, the data processing system may receive a selection of a clinical diagnosis from the computing device. The selection may occur when the user accessing the computing device uses an I/O device to select the clinical diagnosis from the clinical diagnosis or diagnoses that are displayed on the user interface. Upon receiving the selection, the computing device may transmit an indication of the selection to the data processing system including an identification of the selected clinical diagnosis.

The data processing system may receive the indication of the selection and, at step 528, store the indication of the selection in memory. In doing so, in some embodiments, the data processing system may store the indication with the feature vector and/or the data that was used to generate the feature vector in memory. In some embodiments, the data processing system may store the indication in the profile of the patient from which the clinical data was retrieved. Accordingly, the data processing system may later retrieve the indication and/or the associated data or feature vector and maintain a record of all selected clinical diagnoses the data processing system has received for the patient and use such data for training.

At step 530, the data processing system may determine if the selection is being used for training the model (e.g., the model that predicted the clinical diagnoses). The data processing may make this determination by identifying an input the data processing system received from the computing device that made the selection or from an administrator computing device.

If the data processing system identifies an input indicating the received selection and the corresponding data that was used to predict confidence scores for the plurality of medical diagnoses, at step 532, the data processing system may label the feature vector (e.g., the feature vector that was used to generate the confidence scores for the clinical diagnoses) with the selection (e.g., indicate the selected medical diagnosis is the ground truth).

After labeling the feature vector, at step 534, the data processing system may train the model that predicted the confidence scores for the medical diagnoses with the labeled feature vector. The data processing system may do so, for example, by using back propagation techniques on the model where the weights and parameters of the model are adjusted based on differences between the confidence scores and the correct confidence scores. The data processing system may iteratively perform steps 502-534 to train the model to increase the model's accuracy and/or prepare the model for deployment (e.g., for real-time use with the application generating and providing the user interface) upon reaching an accuracy threshold.

In some embodiments, a user at the computing device may train the model using other inputs. For example, the data processing system may predict and transmit a single medical diagnosis to the computing device. In such instances, the user may input whether the prediction was correct or not and the data processing system may adjust the weights of the model according to the input. In another example, the user may input correct confidence scores for the rendered medical diagnoses. The data processing system may use the correct confidence scores to train the model for more accurate predictions. The user may input any type of data to train the model.

If the data processing system determines at step 530 that the feature vector is not being used for training, at step 536, the data processing system may select a treatment plan (e.g., a plan to cure or help alleviate symptoms of the medical diagnosis for the patient) based on the selected clinical diagnosis. The data processing system may select the treatment plan from a database based on an identification of the treatment plan in the database matching the selected clinical diagnosis. In some embodiments, the data processing system may select the treatment plan based on the diagnosis and further clinical data about the patient (e.g., demographic data or symptom data). The data processing system may compare any combination of such data to data in the database and identify the treatment plan with matching values at or above a threshold.

At step 538, the data processing system may transmit a file containing the treatment plan to the computing device that selected the treatment plan. The data processing system may insert the treatment plan into the file and transmit the file to the computing device. The computing device may receive the file and present the treatment plan from the file on the user interface to the user accessing the computing device. In some embodiments, the data processing system may identify a computing device or account associated with the patient and transmit the file to the computing device or account of the patient so the patient has access to the treatment plan. In this way, the data processing system may use a combination of machine learning techniques and separately stored data to accurately identify a treatment plan for a patient.

In some embodiments, the data processing system may use audio and video data the data processing system receives for a clinical encounter to aid a physician of the clinical encounter in diagnosing a patient. For example, the data processing system may receive (e.g., from an on-site video camera) audio and video data of a clinical encounter with a patient having a conversation with a physician. The data processing system may receive the data in a live feed as the patient and the physician are participating in the clinical encounter, in some cases such that both the physician and the patient are depicted in the video data. The data processing system may render the video data and spoken words of the audio data on a user interface of a computing device being accessed by the physician. In some embodiments, the data processing system may use the systems and methods described herein to render translated words of the audio data to the physician to enable the physician to understand what the patient is saying even if the patient is speaking a different language than the physician. The data processing system may extract words from the clinical encounter until reaching a threshold and generate a feature vector from the extracted words and/or clinical data for the patient. The data processing system may insert the feature vector into a model selected based on one or more characteristics of the patient to generate predicted medical diagnoses for the patient. The data processing system may select a subset of the predicted medical diagnoses based on confidence scores for the medical diagnoses and transmit the subset to the physician's computing device. The physician may view the subset of predicted medical diagnoses on the computing device and inform the patient of the diagnosis and/or direct the conversation to discuss options regarding treatment for the clinical diagnosis. In some cases, the physician may input a selection of a clinical diagnosis and the data processing system may select and transmit a treatment plan to the physician based on the selection. In this way, the data processing system may facilitate a clinical encounter to better enable a physician to diagnose a patient.

In some embodiments, the data processing system may generate a real-time tree of questions (e.g., questions of a decision tree) for a physician to ask a patient in a clinical encounter. For example, the data processing system may extract words from audio data the data processing system receives or collects regarding a clinical encounter. The data processing system may use natural language processing techniques to identify and/or extract terms (e.g., stored key words) from the words of the audio data. In some embodiments, the data processing system may do so by identifying words labeled with the patient's name and only using natural language processing system techniques on the identified words, thus ensuring words by the physician do not cause the data processing system to falsely identify a decision tree based on words spoken by the physician that may not be related to a valid line of questioning. In other embodiments, the data processing system may extract terms from all of the spoken words. The data processing system may compare the extracted terms to a database comprising terms that correspond to different question trees (e.g., lists of questions in which questions are asked based on various criteria being met, such as certain words being spoken and/or certain questions being asked). The data processing system may identify the question tree that is associated with one or a threshold number of the extracted terms and select the question tree to use to question the patient.

For example, the data processing system may identify a first question of the question tree and transmit the first question to a computing device being accessed by the physician during the clinical encounter. The physician may read the first question from a user interface on the computer and ask the patient the question. The patient may answer the question and the data processing system may identify the response from new audio data the data processing system receives. The data processing may use natural language processing techniques on the answer to identify a new question from the question tree and transmit the new question to the physician. The data processing system may continue to transmit new questions to the physician in real-time until reaching a final question of the question tree and/or receiving enough spoken words (e.g., words above a threshold) from the patient and/or the physician to generate a feature vector and predict clinical diagnoses for the patient as is described herein.

In some embodiments, the data processing system may operate as a chatbot that uses trees of questions to automatically talk to a patient. Doing so may enable the data processing system to determine a diagnosis for a patient without human intervention. For example, the data processing system may select a tree of questions to ask a patient based on clinical data about the patient and/or words spoken by the patient. The data processing system may do so by comparing the spoken words and/or clinical data to terms in a database that correspond to different trees of questions. The data processing system may identify a tree of questions associated with terms that match the words and/or clinical data based on the comparison. Upon selecting the tree of questions, the data processing system may identify the first question of the decision tree and transmit the question to a computing device being accessed by the patient. The computing device may present the question on a user interface or output audio asking the question. The user may then respond to the question by either typing an answer into the user interface or by saying the answer out loud. The computing device may receive and transmit the response back to the data processing system. Based on the response, the data processing system may select a new question from the question tree. The data processing system may transmit the question back to the computing device. The data processing system may repeat this process until asking the last question of the question tree and/or receiving enough spoken words or answers to make a diagnosis for the patient using the systems and methods described herein.

In some embodiments, the data processing system may operate as a triage tool that is configured to select a set of best fit treatment recommendations for a diagnosis. For example, in addition to or instead of selecting a diagnosis for a patient, the data processing system may select a treatment recommendation based on a risk or illness severity analysis and the diagnosis. To do so, the data processing system may collect multiple different data types for a patient (e.g., medications, therapies, education, lifestyle changes, etc.). The data processing system may collect the data types from a local database or by transmitting requests to external data sources. In some embodiments, the data processing system may analyze the different types of data using a set of patterns or rules to perform a risk or illness severity analysis. The severity analysis may output a severity as a numerical value on a set scale and/or words that correspond to ranges within such a scale (e.g., high, medium, low, etc.). For example, the data processing system may determine an individual that is taking a large number of medications and does not exercise often may have a risk severity, and an individual that exercises every day has a low risk severity. In some embodiments, the data processing system may input the data into a machine learning model that is trained to output a risk or illness severity to perform the analysis. The data processing system may store the determined severity in a profile for the patient. Accordingly, the data processing system may generate a health profile for the patient that may later be used to perform the diagnosis (e.g., used as an input into the machine learning model that outputs a diagnosis or diagnoses) or in combination with a diagnosis to select treatment for the patient.

In some cases, the data processing system may use a combination of a risk or illness severity and a diagnosis to select a treatment for a patient. For example, after triaging the data for a patient to calculate a risk or illness severity and identifying a diagnosis for the patient, the data processing system may use the severity and diagnosis to select or generate the appropriate treatment for the patient. To do so, in some embodiments, the data processing system may first select, from a database, a set of treatments that correspond to a particular diagnosis. From the set of treatments, the data processing system may identify treatments that match or correspond to the risk or illness severity. The data processing system may select treatment plans using any combination of such filtering techniques (e.g., identifying treatment plans that correspond to the risk or illness severity first and then identifying treatments that correspond to the diagnosis or using all of the data at once to query for treatment plans that match the data). The data processing system may transmit the selected treatments to a computing device being accessed by a patient or by a physician treating the patient to provide a more fine-grained treatment plan than systems that do not use such triaging techniques.

In one aspect, the present disclosure is directed to a system for training a model for real-time patient diagnosis. The system may comprise a computer comprising a processor, memory, and a network interface, the processor configured to receive audio data and video data of a clinical encounter, the audio data comprising spoken words by an entity and the video data depicting the entity; retrieve clinical data regarding the entity; execute a model using the words of the audio data and the retrieved clinical data regarding the entity as input, the execution causing the model to output a plurality of clinical diagnoses for the entity; concurrently render the corresponding video data and audio data and the plurality of clinical diagnoses via a computing device associated with a user; and store an indication of a selected clinical diagnosis of the plurality of clinical diagnoses responsive to receiving a selection of the clinical diagnosis at the computing device.

In some implementations, the processor is further configured to label a feature vector comprising the words of the audio data and the retrieved clinical data with the indication of the selected clinical diagnosis; and train the model with the labeled feature vector. In some implementations, the processor is further configured to transcribe the words from the audio data into a text file; and convert the words of the audio data from the text file into a second language from a first language, wherein concurrently rending the video data and the audio data comprises rendering the words in the second language as text on a display of the computing device.

In some implementations, converting the words of the audio data from the text file into the second language comprises converting the words of the audio data into the second language by executing a first translation service, the first translation service selected by the processor from a plurality of translation services by inserting a first text file into each of the plurality of translation services, obtaining a plurality of translated text files each individually associated with a different translation service of the plurality of translation services; receiving one or more indications of errors for each of the plurality of translated text files; calculating an error rate for each of the plurality of translation services based on the one or more indications of errors; and selecting the first translation service responsive to a lowest calculated error rate having an association with the first translation service. In some implementations, the processor is further configured to select a clinical treatment plan based on the selected clinical diagnosis; and transmit a file comprising the selected clinical treatment plan to the computing device.

In some implementations, the processor executing the model causes the model to output a confidence score for each of the plurality of clinical diagnoses, the processor further configured to generate a sequential order of the plurality of clinical diagnoses based on the confidence score for each of the plurality of clinical diagnoses, wherein concurrently rendering the plurality of clinical diagnoses comprises rendering text identifying the plurality of clinical diagnoses in the sequential order on a display of the computing device. In some implementations, the processor executing the model causes the model to output a confidence score for each of the plurality of clinical diagnoses, and wherein the processor is configured to concurrently render the plurality of clinical diagnoses by rendering the confidence score for each of the plurality of clinical diagnoses on a display of the computing device.

In some implementations, the processor is further configured to identify one or more characteristics of the patient from the clinical data, the video data, or the audio data; and select the model from a plurality of models based on the one or more characteristics. In some implementations, the processor is configured to receive the audio data and video data of the clinical encounter by receiving the audio data and video data in real-time during the clinical encounter, and wherein the processor is configured to concurrently render the corresponding video data and audio data and the plurality of clinical diagnoses via the computing device associated with the user by concurrently rendering the corresponding video data and audio data and the plurality of clinical diagnoses in real time during the clinical encounter. In some implementations, the video data further depicts the user.

In some implementations, the processor is further configured to extract a term from the audio data comprising spoken words of the entity; select a decision tree comprising a set of questions based on the extracted term; and sequentially render the set of questions on a display of the computing device during the clinical encounter based on second audio data comprising one or more answers to the set of questions, the answers spoken words by the entity.

In another aspect, the present disclosure is directed to a method for training a model for real-time patient diagnosis, comprising receiving, by a processor, audio data and video data of a clinical encounter, the audio data comprising spoken words by an entity and the video data depicting the entity; retrieving, by the processor, clinical data regarding the entity; executing, by the processor, a model using the words of the audio data and the retrieved clinical data regarding the entity as input to output a plurality of clinical diagnoses for the entity; concurrently rendering, by the processor, the corresponding video data and audio data and the plurality of clinical diagnoses via a computing device associated with a user; and storing, by the processor, an indication of a selected clinical diagnosis of the plurality of clinical diagnoses responsive to receiving a selection of the clinical diagnosis at the computing device.

In some implementations, the method further comprises labeling, by the processor, a feature vector comprising the words of the audio data and the retrieved clinical data with the indication of the selected clinical diagnosis; and training, by the processor, the model with the labeled feature vector. In some implementations, the method further comprises transcribing, by the processor, the words from the audio data into a text file; and converting, by the processor, the words of the audio data from the text file into a second language from a first language, wherein concurrently rending the video data and the audio data via the computing device comprises rendering, by the processor, the words in the second language as text on a display of the computing device.

In some implementations, converting the words of the audio data from the text file into the second language comprises converting, by the processor, the words of the audio data into the second language by executing, by the processor, a first translation service, the first translation service selected by the processor from a plurality of translation services by inserting, by the processor, a first text file into each of the plurality of translation services, obtaining a plurality of translated text files each individually associated with a different translation service of the plurality of translation services; receiving, by the processor, one or more indications of errors for each of the plurality of translated text files; calculating, by the processor, an error rate for each of the plurality of translation services based on the one or more indications of errors; and selecting, by the processor, the first translation service responsive to a lowest calculated error rate having an association with the first translation service.

In some implementations, the method further comprises selecting, by the processor, a clinical treatment plan based on the selected clinical diagnosis; and transmitting, by the processor, a file comprising the selected clinical treatment plan to the computing device. In some implementations, executing the model causes the model to output a confidence score for each of the plurality of clinical diagnoses, and the method further comprises generating, by the processor, a sequential order of the plurality of clinical diagnoses based on the confidence score for each of the plurality of clinical diagnoses, wherein concurrently rendering the plurality of clinical diagnoses comprises rendering, by the processor, strings identifying the plurality of clinical diagnoses in the sequential order on a display of the computing device. In some implementations, executing the model causes the model to output a confidence score for each of the plurality of clinical diagnoses, and wherein concurrently rendering the plurality of clinical diagnoses comprises rendering, by the processor, the confidence score for each of the plurality of clinical diagnoses on a display of the computing device. In some implementations, the method further comprises identifying, by the processor, one or more characteristics of the patient from the clinical data, the video data, or the audio data; and selecting, by the processor, the model from a plurality of models based on the one or more characteristics.

In another aspect, the present disclosure is directed to a non-transitory computer readable medium. The non-transitory computer readable medium may include encoded instructions that, when executed by a processor of a computer, cause the computer to receive audio data and video data of a clinical encounter, the audio data comprising spoken words by an entity and the video data depicting the entity; retrieve clinical data regarding the entity; execute a model using the words of the audio data and the retrieved clinical data regarding the entity as input to output a plurality of clinical diagnoses for the entity; concurrently render the corresponding video data and audio data and the plurality of clinical diagnoses via a computing device associated with a user; and store an indication of a selected clinical diagnosis of the plurality of clinical diagnoses responsive to receiving a selection of the clinical diagnosis at the computing device.

The various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of this disclosure or the claims.

Embodiments implemented in computer software may be implemented in software, firmware, middleware, microcode, hardware description languages, or any combination thereof. A code segment or machine-executable instructions may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc., may be passed, forwarded, or transmitted via any suitable means including memory sharing, message passing, token passing, network transmission, etc.

The actual software code or specialized control hardware used to implement these systems and methods is not limiting of the claimed features or this disclosure. Thus, the operation and behavior of the systems and methods were described without reference to the specific software code being understood that software and control hardware can be designed to implement the systems and methods based on the description herein.

When implemented in software, the functions may be stored as one or more instructions or code on a non-transitory computer-readable or processor-readable storage medium. The steps of a method or algorithm disclosed herein may be embodied in a processor-executable software module, which may reside on a computer-readable or processor-readable storage medium. A non-transitory computer-readable or processor-readable media includes both computer storage media and tangible storage media that facilitate transfer of a computer program from one place to another. A non-transitory processor-readable storage media may be any available media that may be accessed by a computer. By way of example, and not limitation, such non-transitory processor-readable media may comprise RAM, ROM, EEPROM, CD-ROM or other optical disk storage, magnetic disk storage or other magnetic storage devices, or any other tangible storage medium that may be used to store desired program code in the form of instructions or data structures and that may be accessed by a computer or processor. Disk and disc, as used herein, include compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm may reside as one or any combination or set of codes and/or instructions on a non-transitory processor-readable medium and/or computer-readable medium, which may be incorporated into a computer program product.

The preceding description of the disclosed embodiments is provided to enable any person skilled in the art to make or use the embodiments described herein and variations thereof. Various modifications to these embodiments will be readily apparent to those skilled in the art, and the principles defined herein may be applied to other embodiments without departing from the spirit or scope of the subject matter disclosed herein. Thus, the present disclosure is not intended to be limited to the embodiments shown herein but is to be accorded the widest scope consistent with the following claims and the principles and novel features disclosed herein.

While various aspects and embodiments have been disclosed, other aspects and embodiments are contemplated. The various aspects and embodiments disclosed are for purposes of illustration and are not intended to be limiting, with the true scope and spirit being indicated by the following claims.

### EXAMPLES

### Example 1

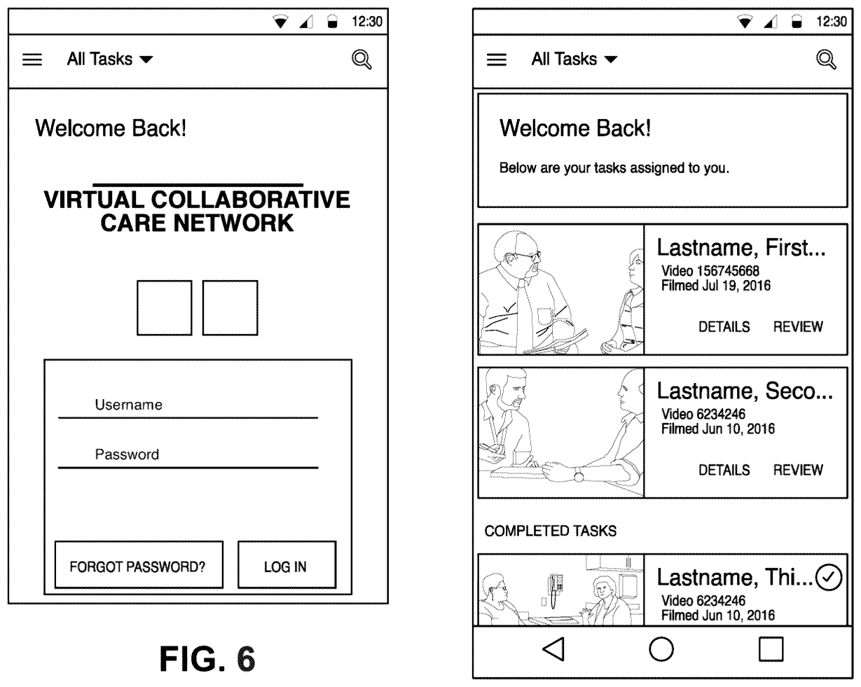

FIGS. 6-17 illustrate graphical user interfaces generated and displayed in a multi-linguistic diagnostic and screening analysis system, according to an embodiment.

In example 1, at FIG. 6, a healthcare provider may log into the clinic end user device. The graphical user interface (GUI) may show a series of assignments directed to the health care provider to generate a clinical diagnosis and/or treatment plan. Each assignment may correspond to a different patient. The assignment may include data such as the patient's health information, patient socioeconomic information, and videos taken of patients. In some embodiments, a third-party may take the interview with the patient to be later viewed asynchronously by the healthcare provider.

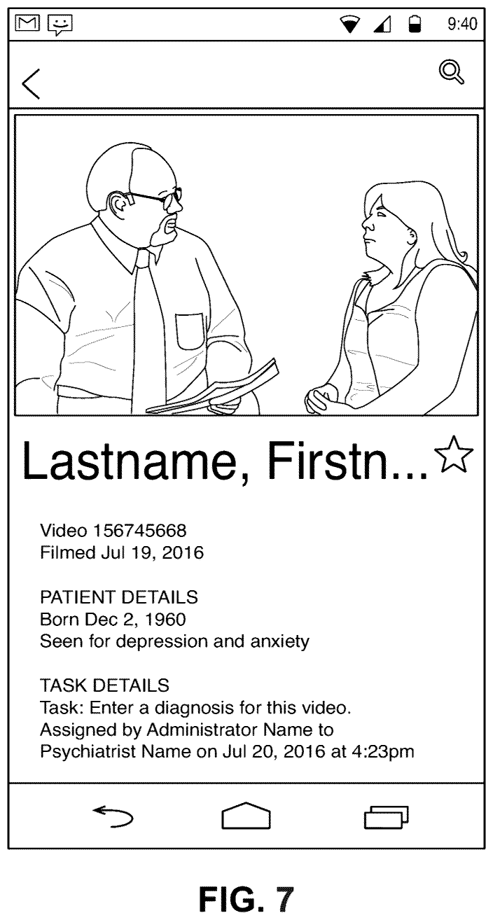

FIG. 7 shows an alternate provider view in the GUI. The GUI may show the video along with patient details such as date of birth and the reason the patient sought treatment. The GUI may also show the task the clinician is asked to perform, the administrator who assigned the task, and the name of the clinician (in this case a psychiatrist) assigned the task.

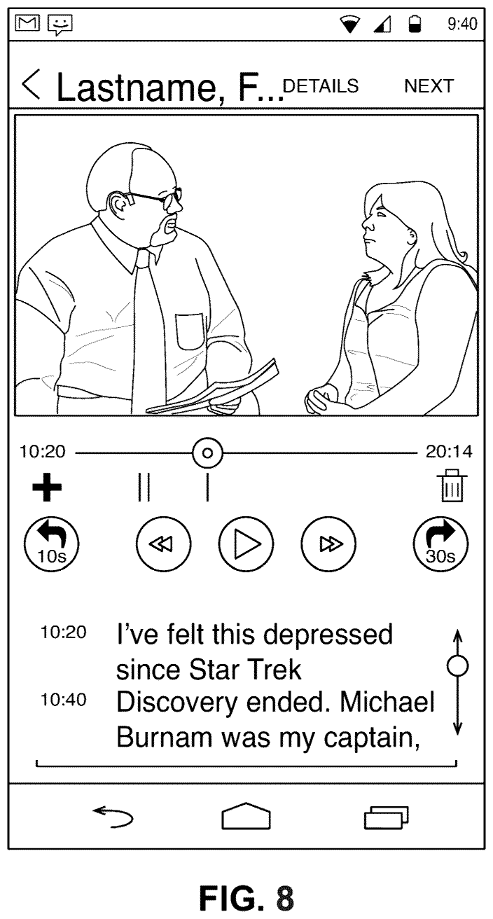

FIG. 8 shows an alternate provider view of the GUI. The GUI may show the video along with the transcription or translation of the spoken text from the video. For instance, if the provider preferred language and the interview language is the same, the GUI may show a direct transcription of the spoken text, for instance as subtitles. If the provider preferred language is different from the language the interview is held in, the system may perform a translation of the interview and show the translated text as subtitles or as auditory “dubbing”.

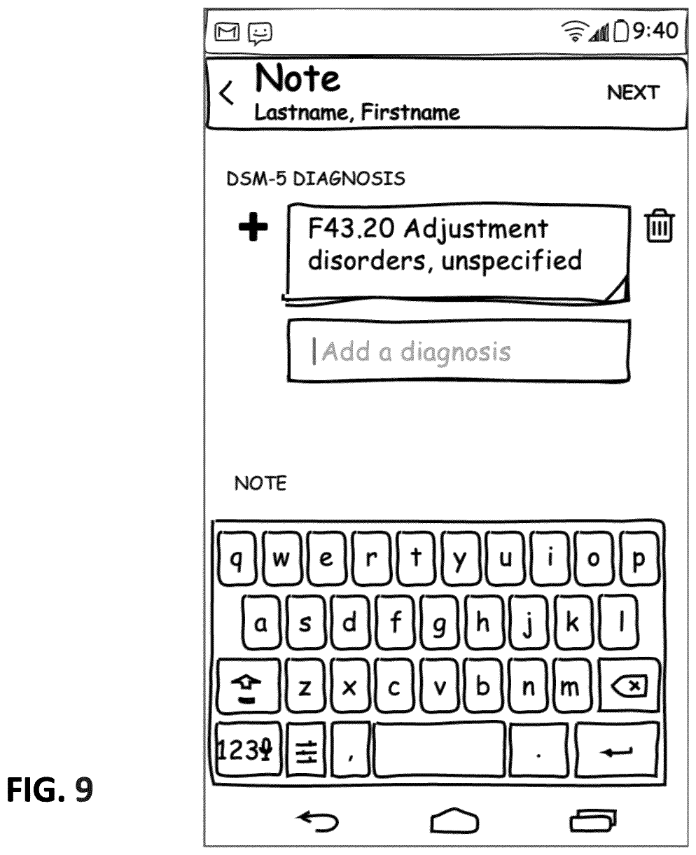

FIG. 9 shows an alternate provider view of the GUI. The GUI may provide a place for the clinician to enter notes, for instance a diagnosis of the patient, or other pertinent details.

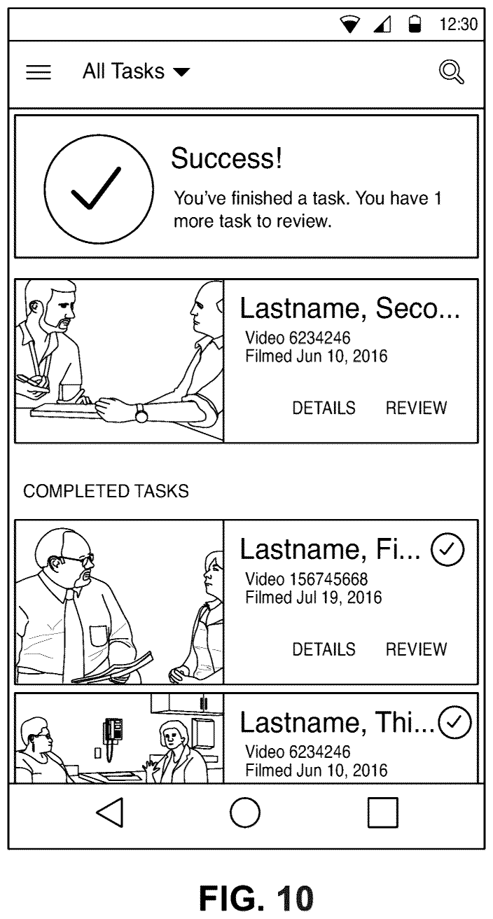

FIG. 10 shows an alternate provider view of the GUI. The GUI may show completed tasks, along with task that still need completing along with the ability to drill down into details of the completed assessment.

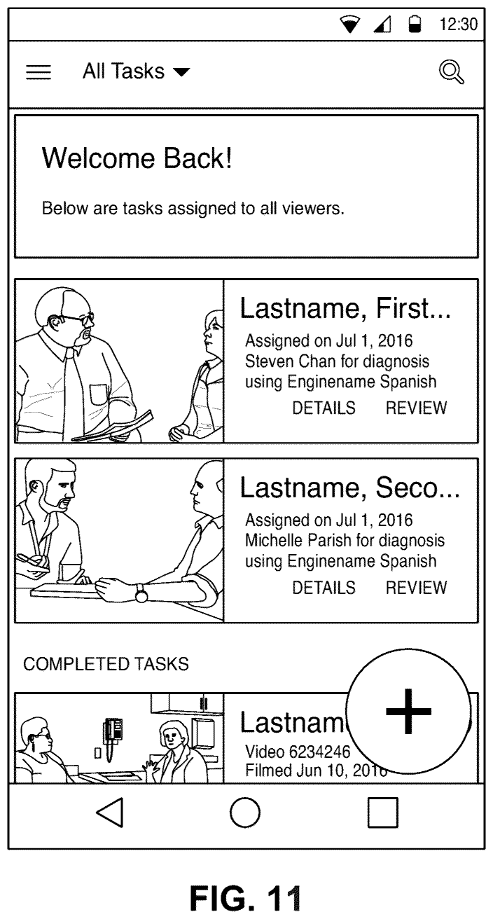

FIG. 11 shows an administrator view of the GUI. The GUI may show all the tasks that have been assigned and completed, as well as tasks that are not yet assigned.

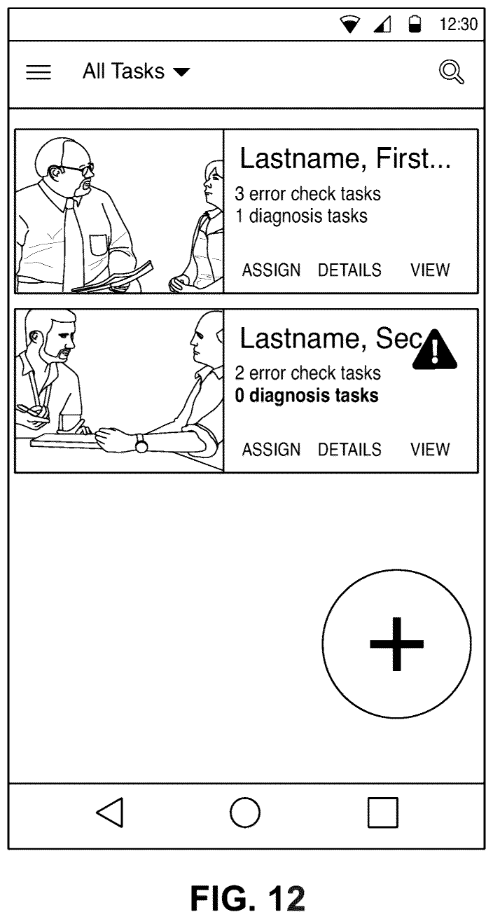

FIG. 12 shows an alternate administrator view of the GUI. The GUI may show all the tasks that need to be assigned for each patient video as well as the type of assignment. For instance, the GUI may show that for one patient video, there are three error check tasks and one diagnosis task that needs to be completed. Error check tasks may be tasks such as a check of the machine translation of the video or a machine diagnosis. A diagnosis task may include the task of diagnosis of the patient by a (human) healthcare provider.

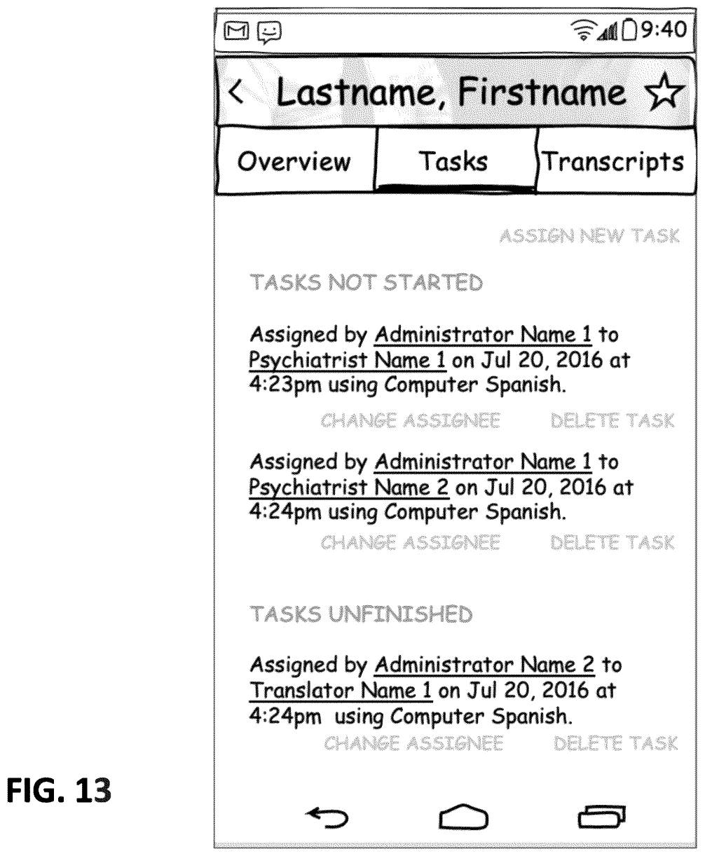

FIG. 13 shows an alternate administrator view of the GUI. The GUI may show a list of assignments, the administrator who assigned the task, and the provider or translator the task was assigned to. The GUI may also show the degree of completion of each task.

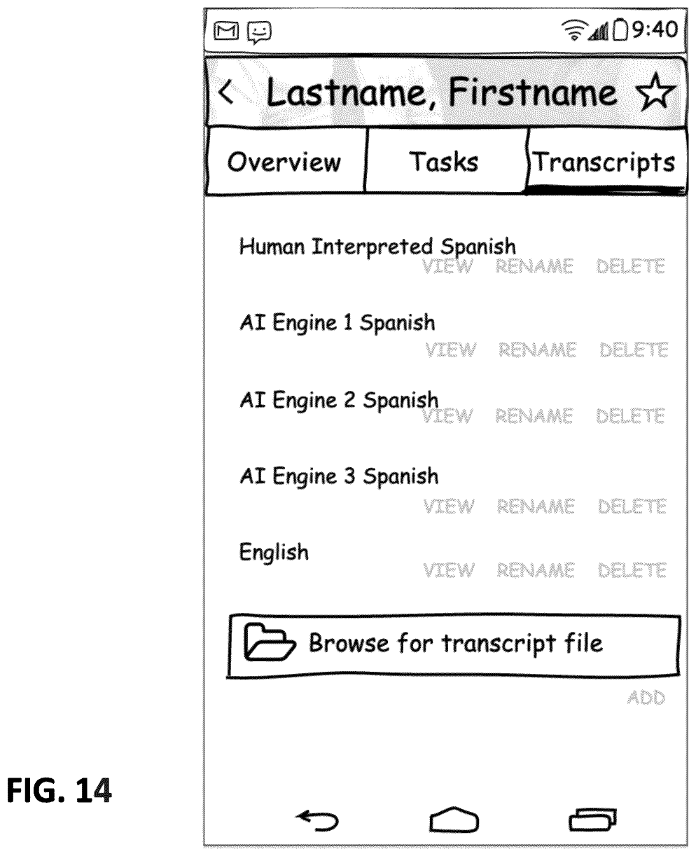

FIG. 14 shows an alternate view for both providers and administrators of the GUI. The GUI may show a list of transcriptions and/or translations of the audio of the video available for each video.

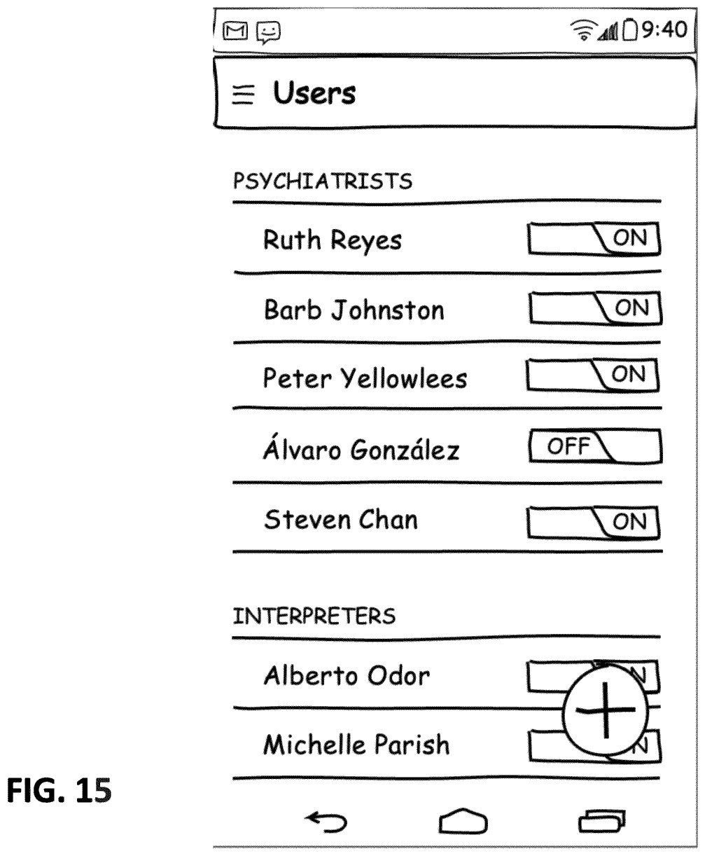

FIG. 15 shows an alternate view for administrators of the GUI. The GUI may show a list of available healthcare providers and interpreters for assignment.

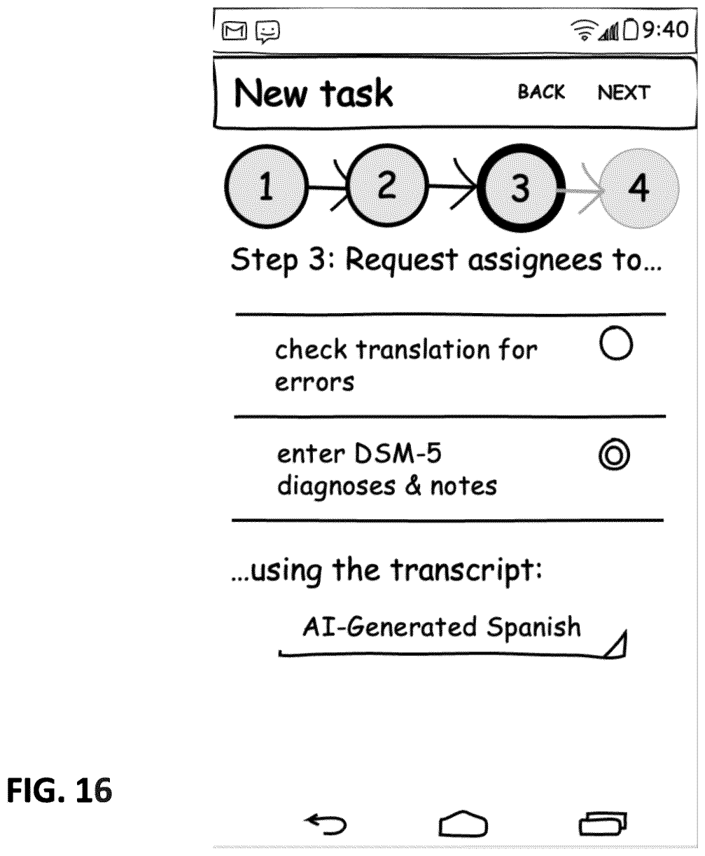

FIG. 16 shows an alternate view for administrators of the GUI. The GUI may show the steps that need to be taken to complete the diagnosis of the patient and the steps that have already been completed.

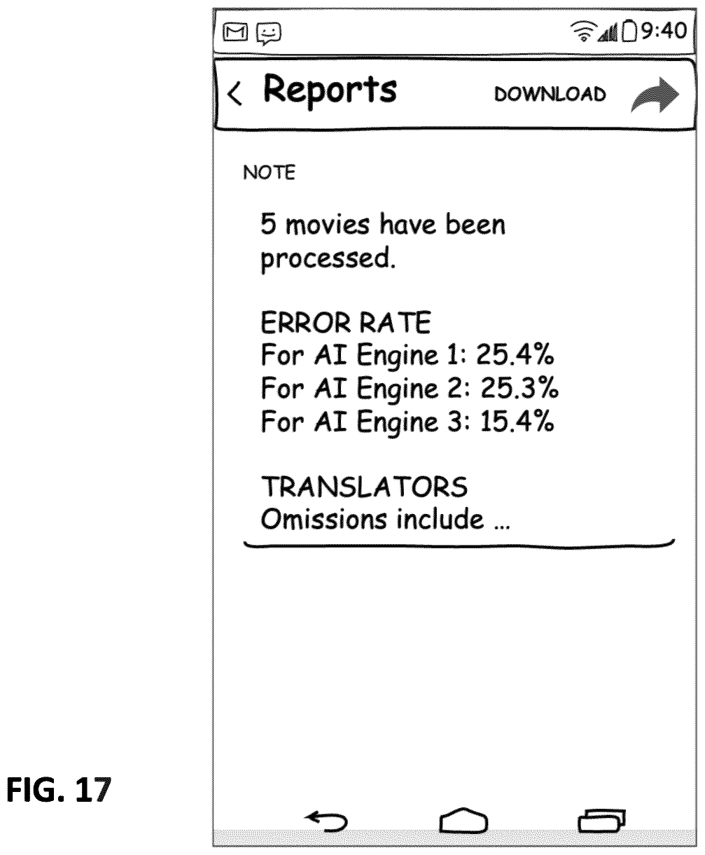

FIG. 17 shows an alternate view for administrators of the GUI. The GUI may provide information on the error rate of various translation methods. This may be determined using the analytics server trained on a corpus or using human translation.

### Example 2

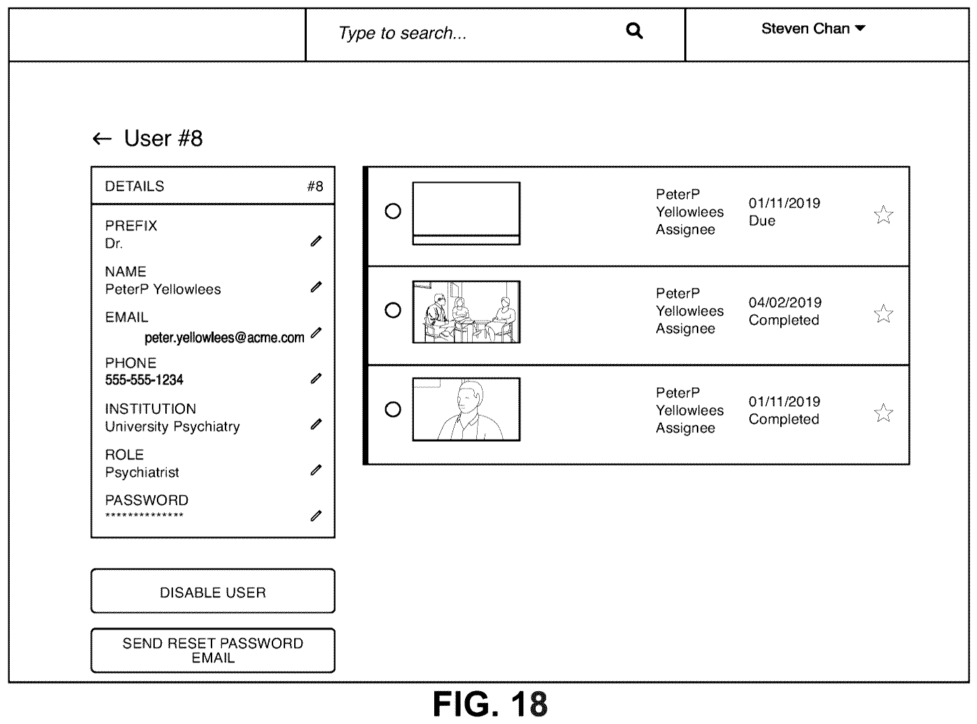

FIGS. 18-25 illustrate graphical user interfaces generated and displayed in a multi-linguistic diagnostic and screening analysis system, according to an embodiment

FIG. 18 shows a provider view of the GUI according to one embodiment. The provider may be able to see a list of assigned tasks as well as the assigner and completion status.

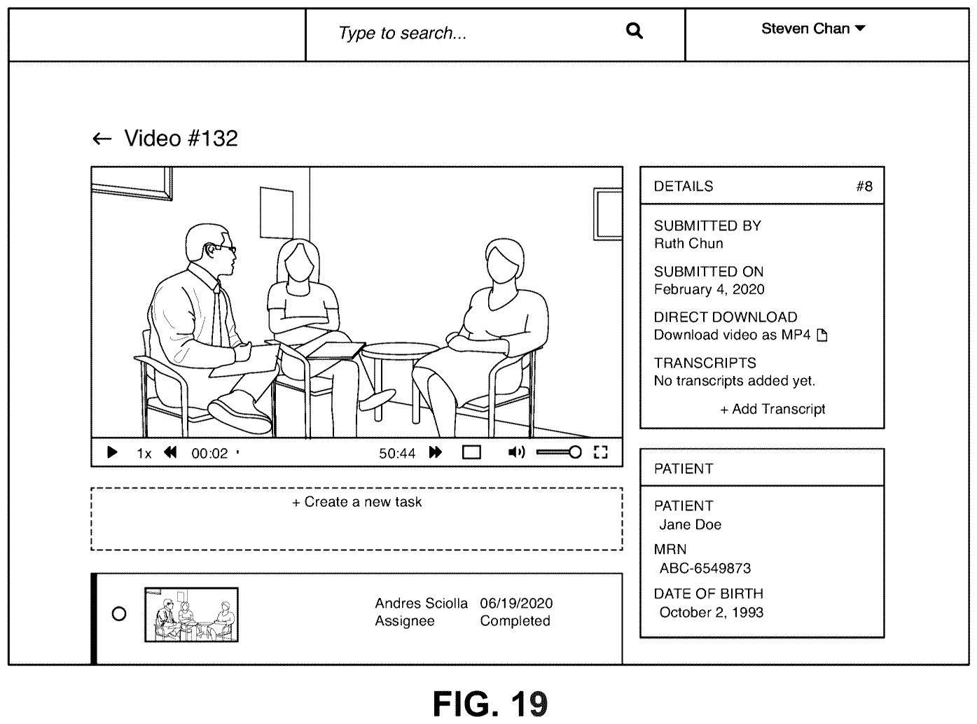

FIG. 19 shows an alternate provider view. The provider may be able to see a video of an interview of the patient taken by a third party or computer interviewer. The GUI may also display the submitter and date of the interview and method to download and/or parse the video. The GUI may show available transcripts or translations and may give the provider the ability to add a transcript using an automated translation system. The GUI may additionally show details regarding the patient such as the date of birth, comorbidities, or other factors.

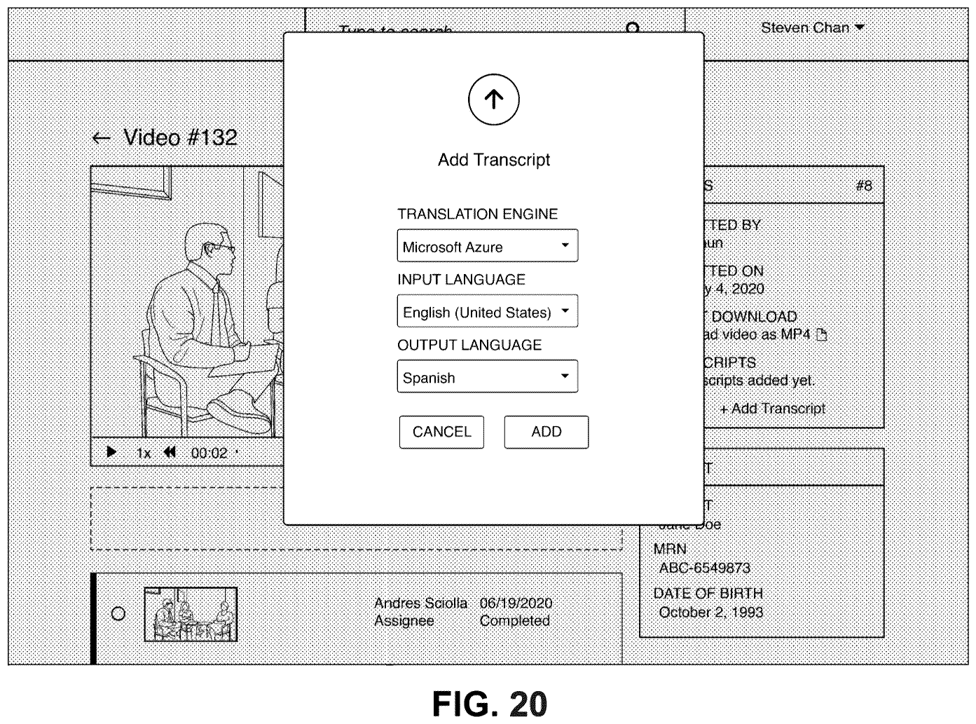

FIG. 20 shows an alternate provider view. The provider may elect to get a translation of the video. The GUI may show the provider available translation languages. The provider may select the language of the interview and the desired language of the translation.

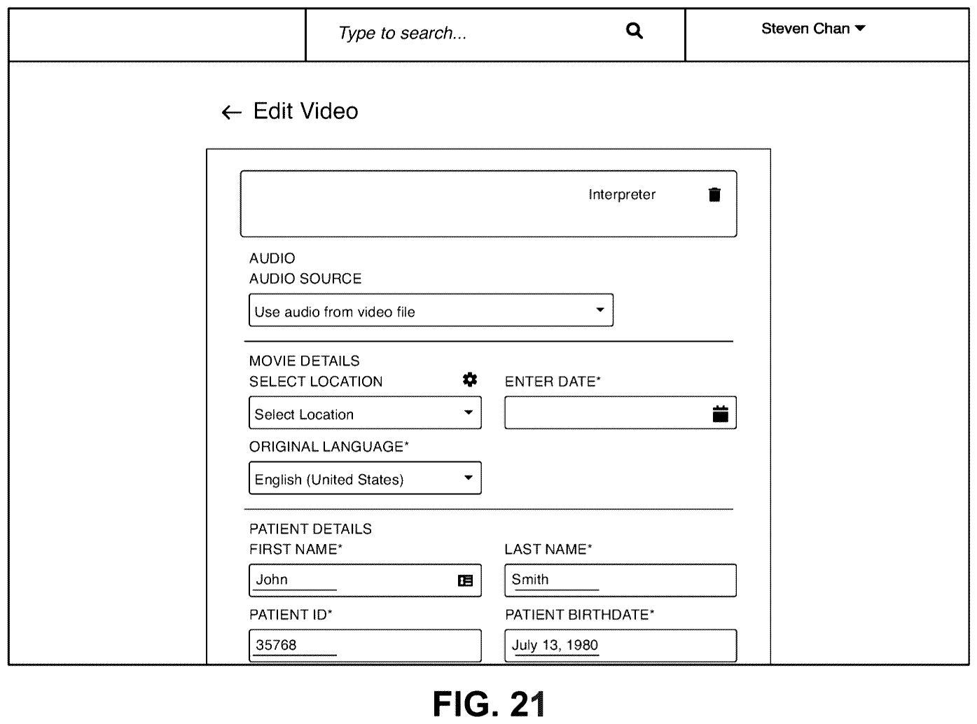

FIG. 21 allows the provider to enter details regarding the audio source from the video file and the location of the movie. In other embodiments, the analytical system may automatically determine these inputs.

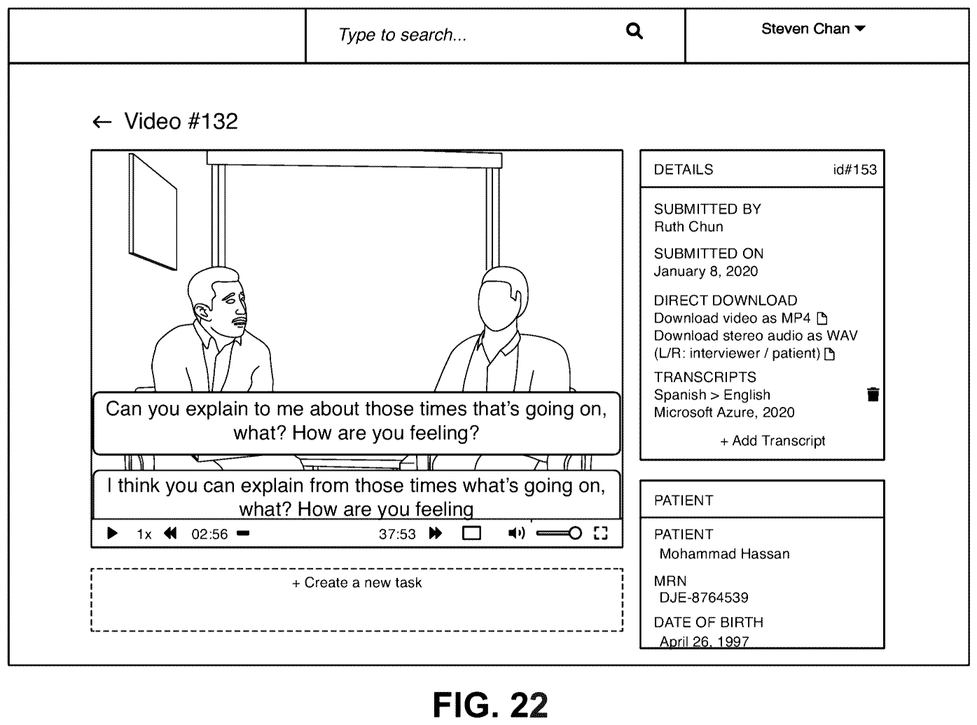

FIG. 22 shows an alternate provider view where the provider has elected to have the audio transcribed into subtitles which overlay the video. Alternately, the provider may elect to have a whole transcription on a separate screen or subtitles beneath the video.

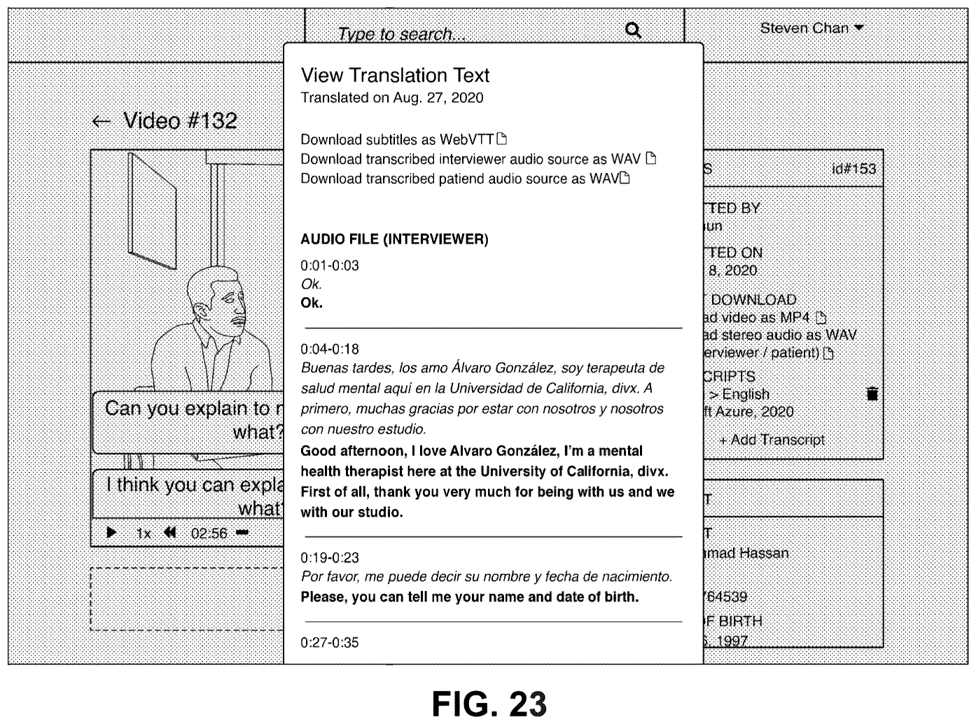

FIG. 23 shows an alternate provider view where the provider has elected to have the audio translated from the language of the video to the provider's preferred language. In this case, the interview was held in Spanish and the provider has elected to have the video translated into English for review. In some instances, the translation text may appear in a different box or screen from the video. In other instances, the translation text may appear as subtitles below the video or which overlay the video. In other instances, the translation may be in computer generated audio “dubbed” over the video.

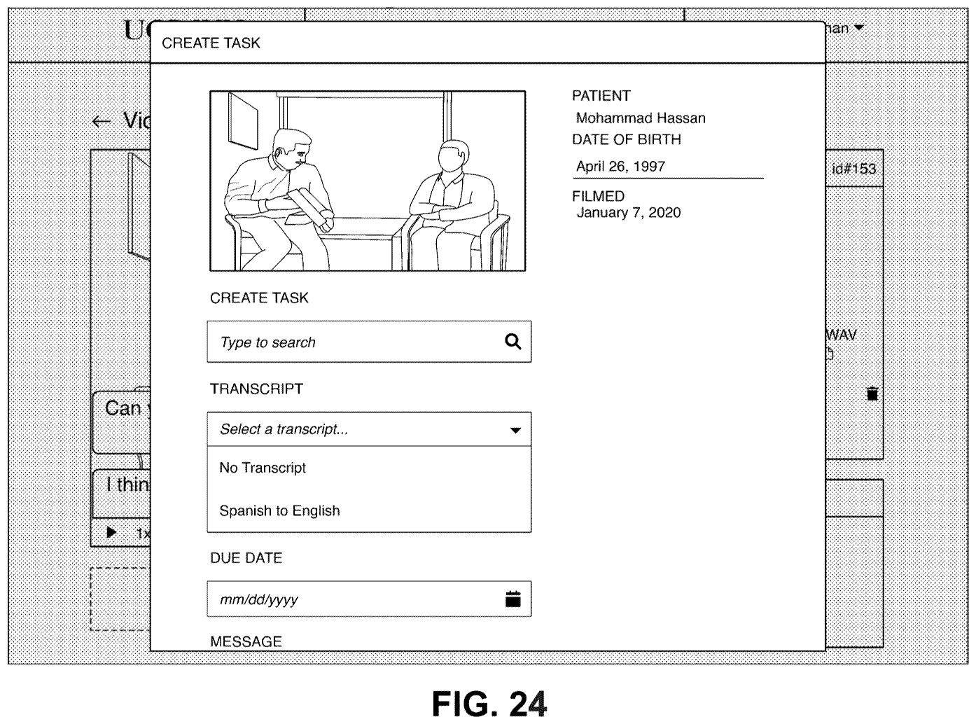

FIG. 24 shows an administrator or healthcare provider view where the administrator or healthcare provider assigns a task to a human transcriber or translator to transcribe or translate the video, or to check the automatically generated transcription or translation.

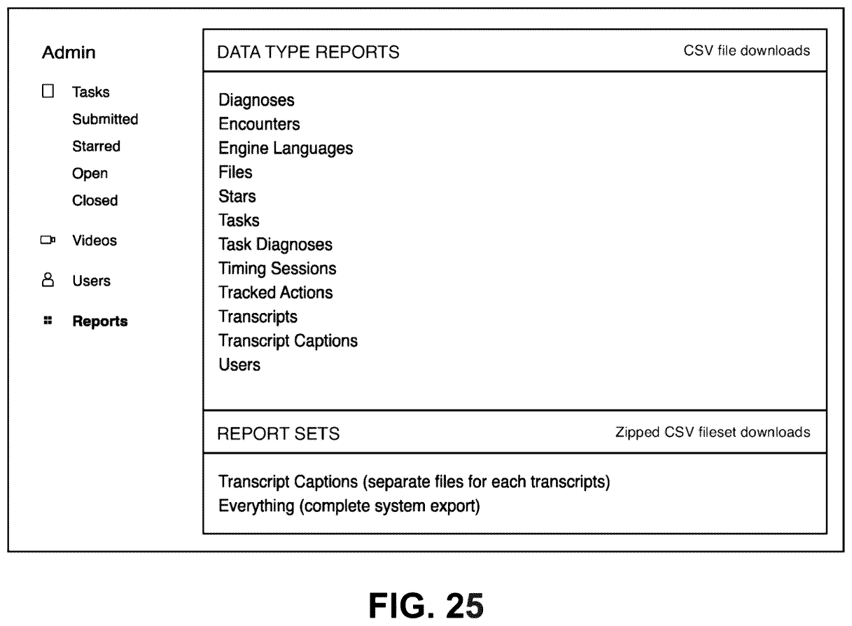

FIG. 25 shows an administrator view where the administrator can view data reports for optimization of the procedure or data analytics server.

### Example 3

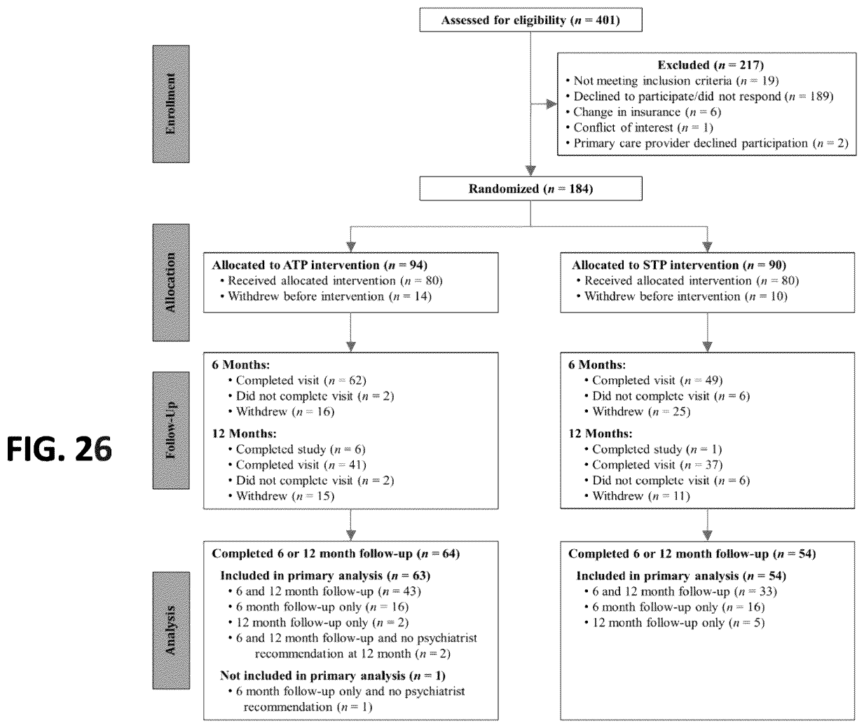

FIG. 26 illustrates the results of an asynchronous multi-linguistic diagnostic and screening analysis system, according to one embodiment. FIG. 26 shows the enrollment, allocation, and follow-up of a comparative study between Asynchronous treated patient (ATP), or patients treated by a provider after the interview has completed, and Synchronous treated patients (STP), or patients treated in real time.

Thirty-six primary care providers (PCP) from 3 primary care clinics referred a heterogeneous sample of 401 treatment seeking adult patients with non-urgent psychiatric disorders. A total of 184 (94 ATP, 90 STP) English and Spanish speaking participants (20% Hispanic) were enrolled and randomized; 160 (80 ATP, 80 STP) completed baseline evaluations. Patients were treated by their PCPs using a collaborative care model in consultation with University of California Davis Health telepsychiatrists who consulted with the patients every six months for up to two years using ATP or STP. Primary (clinician rated Clinical Global Impressions scale [CGI] and the Global Assessment of Functioning [GAF]) and secondary (patients' self-reported physical and mental health, and depression) outcomes were assessed every six months.

ATP assessments were conducted at six-month intervals by an ATP trained clinician who spoke the patient's primary language, either English or Spanish. This interview was video recorded using HIPAA-compliant security systems and protocols. For each ATP assessment, the clinician updated a standardized electronic form to capture notes about clinically relevant or important material observed during the interview. These notes were usually completed the day of the ATP interview so that study psychiatrists had rapid access to the entire interview video, the clinician's interview notes, and previous medical and sometimes psychiatric assessments of the patient already recorded in their EMR. Each patient's psychiatrist provided the patient's PCP with a written assessment and psychiatric treatment plan. The PCP also had continuing access to this psychiatrist by phone or email between the study consultations for up to two years.

The clinical workflow process for the STP arm was similar to the ATP arm; except that ATP recorded assessments were replaced by live real-time STP assessments conducted by a psychiatrist who spoke the patient's preferred language, either English or Spanish. After the STP consultation the psychiatrist provided the patient's PCP with a written assessment and treatment plan in their EMR and was available for future contact by phone or email as necessary.

A demographic questionnaire was administered at baseline to collect sociodemographic information. Participants were clinically assessed in both study arms at 6-month intervals (baseline, 6 months, 12 months, 18 months, and 24 months), with the primary outcome measures completed by the treating psychiatrists. All other study questionnaires assessing self-reported outcomes were collected every 6 months by research assistants either by phone, or via paper or electronic surveys depending on participants' preferences.

The primary outcomes were derived from the psychiatrists report and included the Clinical Global Impressions scale (CGI) and the Global Assessment of Functioning (GAF) The CGI is a 3-item 7-point observer-rated scale that measures illness severity, global improvement or change and therapeutic response. The CGI is considered a robust measure with established validity in inpatient, outpatient, and clinical trial settings. The CGI severity of illness and improvement scales are commonly used in non-drug trial settings. We used the CGI severity of illness scale, scored from 1 [normal] to 7 [among the most extremely ill]. The GAF is a widely used rating scale to assess impairment among patients with psychiatric disorders. The GAF assesses the level of psychological, social, and occupational functioning on a 1-100 scale, with higher levels indicating better functioning.

Secondary outcomes focused on patient self-report and included the 12-Item Short Form Health Survey's (SF-12), Physical Health Component Summary (PHS-12) and Mental Health Component Summary (MHS-12) scores (both scored from 0 to 100, with higher scores indicating better health) and the Patient Health Questionnaire-9 (PHQ-9). The PHQ-9 is a well validated depression scale with scores derived as the sum of 9 items (each scored from 0 [not at all] to 3 [nearly every day]; scale range 0 to 27) based directly on the diagnostic criteria for major depressive disorder in the DSM-IV (Diagnostic and Statistical Manual Fourth Edition).

FIG. 26 depicts the flow of patients from screening through the primary endpoint, the 12-month follow-up. Of 401 patients assessed for eligibility, 184 were enrolled and randomized to the ATP (n=94) or STP (n=90) intervention. Of the 184 randomized participants, 18 (11 ATP, 7 STP) were consented to 12-month follow up and 24 (14 ATP, 10 STP) withdrew before the baseline visit. Reasons for withdrawal before baseline included insurance changes (n=2), decline to participate (n=7), and loss to follow-up (n=15).

Table 1 compares the demographic and clinical characteristics for the 160 participants who completed the baseline visit and the 24 who did not.

TABLE 1
Baseline demographic and clinical characteristics of participants
who completed baseline visits

| | ATP^{b} | STP^{c} | Total |
|---|---|---|---|
| Characteristic^{a} | (n = 80) | (n = 80) | (n = 160) |
| Age (years), mean (SD^{d}) | 53.0 (14.0) | 52.2 (14.6) | 52.6 (14.3) |
| Number of Axis I diagnoses, | 2.4 (1.0) | 2.4 (1.0) | 2.4 (1.0) |
| mean (SD) | | | |
| Screening PHQ-9 score^{e,f,g}, | 13.9 (6.6) | 13.1 (5.9) | 13.5 (6.3) |
| mean (SD) | | | |
| Screening PHQ-9 category^{g}, | | | |
| n (%) | | | |
| 0-4, Nondepressed | 5 (6.3) | 8 (10.3) | 13 (8.3) |
| 5-9, Mild depression | 18 (22.8) | 16 (20.5) | 34 (21.7) |
| 10-14, Moderate depression | 21 (26.6) | 23 (29.5) | 44 (28.0) |
| ≥15, Moderately severe to | 35 (44.3) | 31 (39.7) | 66 (42.0) |
| severe depression | | | |
| Primary diagnosis, n (%) | | | |
| Mood disorder | 54 (67.5) | 54 (67.5) | 108 (67.5) |
| Anxiety disorder | 16 (20.0) | 16 (20.0) | 32 (20.0) |
| Substance abuse | 2 (2.5) | 1 (1.3) | 3 (1.9) |
| Other | 8 (10.0) | 9 (11.3) | 17 (10.6) |
| Female, n (%) | 58 (72.5) | 53 (66.3) | 111 (69.4) |
| Hispanic ethnicity, n (%) | 15 (18.8) | 15 (18.8) | 30 (18.8) |
| Education, n (%) | | | |
| Graduate high school or less | 22 (27.5) | 18 (22.5) | 40 (25.0) |
| Some college/2-year college | 32 (40.0) | 40 (50.0) | 72 (45.0) |
| College/Graduate school | 26 (32.5) | 22 (27.5) | 48 (30.0) |
| Marital status^{h}, n (%) | | | |
| Married/living with someone | 39 (51.3) | 39 (52.7) | 78 (52.0) |
| Otherⁱ | 37 (48.7) | 35 (47.3) | 72 (48.0) |
| Current psychiatric treatment^{j}, | 31 (39.7) | 34 (42.5) | 65 (41.1) |
| n (%) | | | |
| Current psychotropic | 64 (83.1) | 66 (82.5) | 130 (82.8) |
| medication^{k}, n (%) | | | |
| Language of the interview | | | |
| English | 71 (88.8) | 70 (87.5) | 141 (88.1) |
| Spanish | 9 (11.3) | 10 (12.5) | 19 (11.9) |
| Study clinic, n (%) | | | |
| Auburn | 44 (55.0) | 43 (53.8) | 87 (54.4) |
| J Street (Sacramento) | 17 (21.3) | 19 (23.8) | 36 (22.5) |
| Communicare | 19 (23.8) | 18 (22.5) | 37 (23.1) |

Due to rounding percentages might not sum to 100.
^{a}There were no significant differences between the two intervention groups for any
characteristic.
^{b}ATP: Asynchronous Telepsychiatry.
^{c}STP: Synchronous Telepsychiatry.
^{d}SD: standard deviation.
^{e}PHQ-9: Patient Health Questionnaire-9.
^{f}Range 0-27, higher is more depressed.
^{g}Data missing = 1 in ATP group and 2 in STP.
^{h}Data missing = 4 in ATP group and 6 in STP.
ⁱIncludes widowed, divorced or annulled, separated, and never married.
^{j}Data missing = 2 in ATP group.
^{k}Data missing = 3 in ATP group.

The two groups were similar in socio-demographic characteristics and depression symptoms, but participants who completed the baseline visit were more likely to be receiving current outpatient psychotherapy for a psychiatric condition (41.1% vs. 20.8%, P=0.06) and to be using psychotropic medication (82.8% vs. 50.0%, P<0.001) than those who did not complete baseline visits. Interestingly only 1 of these 160 patients who completed a baseline visit was seeing an outpatient psychiatrist, with the rest all being treated in primary care.

Table 2 summarizes mean trajectories and changes from baseline in the 2 arms for the clinician ratings (CGI and GAF) and the results of mixed-effects models for the primary analysis. For both ratings, both ATP and STP arms improved at 6 and 12 months as compared to baseline. Patients in both arms had about 1 point improvements on CGI at 6 months follow-up (estimated difference from baseline-0.7, 95% CI −1.0 to 0.4, P<0.001, for ATP and −0.9, 95% CI −1.2 to −0.6, P<0.001, for STP) and these improvements were maintained at 12-months (estimated difference from baseline-0.8, 95% CI −1.1 to −0.5, P<0.001, for ATP and −1.2, 95% CI −1.5 to −0.9, P<0.001, for STP). The results for GAF were similar, with both groups improving by about 3 points at 6-months (estimated difference from baseline 2.7, 95% CI 1.1 to 4.4, P=0.002, for ATP and 3.3, 95% CI 1.4 to 5.1, P<0.001, for STP) and by about 5 points at 12-months follow-up (estimated difference from baseline 4.7, 95% CI 2.8 to 6.5, P<0.001, for ATP and 5.2, 95% CI 3.2 to 7.2, P<0.001, in STP). None of the interactions between the intervention arm and follow-up times were significant (all Ps>0.07), suggesting that the level of improvement was similar for the two groups.

TABLE 2
PRIMARY OUTCOMES: CLINICIAN RATINGS AT BASELINE AND 6 AND 12-MONTH
FOLLOW-UP FOR THE 117 PATIENTS INCLUDED IN PRIMARY ANALYSIS

| | | | | CGI | GAF |
|---|---|---|---|---|---|
| | | CGI^{a,b} | GAF^{c,d} | Estimate, | Estimate, |
| | n | Mean (SD^{e}) | Mean (SD) | Mean (95% CI)^{f} | Mean (95% CI)^{f} |
| ATP^{g} | | | | | |
| Mean Trajectory | | | | | |
| Baseline | 63 | 3.9 (0.9) | 59.7 (10.8) | — | — |
| Follow-up at 6 months | 61 | 3.2 (1.0) | 62.4 (11.9) | — | — |
| Follow-up at 12 months | 45 | 3.1 (1.1) | 63.7 (13.0) | — | — |
| Change from baseline | | | | | |
| 6 months vs. baseline | 61 | −0.7 (1.0) | 2.8 (6.3) | −0.7 (−1.0 to −0.4) | 2.7 (1.1 to 4.4) |
| 12 months vs. baseline | 45 | −0.8 (1.2) | 4.4 (8.7) | −0.8 (−1.1 to −0.5) | 4.7 (2.8 to 6.5) |
| STP^{h} | | | | | |
| Mean Trajectory | | | | | |
| Baseline | 54 | 4.2 (1.0) | 57.6 (10.2) | — | — |
| Follow-up at 6 months | 49 | 3.3 (1.0) | 60.7 (11.0) | — | — |
| Follow-up at 12 months | 38 | 3.0 (1.0) | 61.8 (12.2) | — | — |
| Change from baseline | | | | | |
| 6 months vs. baseline | 49 | −0.9 (1.0) | 2.9 (6.4) | −0.9 (−1.2 to −0.6) | 3.3 (1.4 to 5.1) |
| 12 months vs. baseline | 38 | −1.2 (1.0) | 5.1 (6.3) | −1.2 (−1.5 to −0.9) | 5.2 (3.2 to 7.2) |
| ATP vs. STP, differences at | | — | — | −0.3 (−0.6 to 0.1) | 0.9 (−2.1 to 4.0) |
| baseline | | | | | |
| ATP vs. STP, differences at | | — | — | −0.1 (−0.4 to 0.3) | 0.4 (−2.8 to 3.5) |
| follow-up at 6 months | | | | | |
| ATP vs. STP, differences at | | — | — | 0.1 (−0.3 to 0.5) | 0.4 (−2.9 to 3.7) |
| follow-up at 12 months | | | | | |
| ATP vs. STP, differences in | | — | — | 0.2 (−0.2 to 0.6) | −0.6 (−3.1 to 1.9) |
| follow-up at 6 months vs. | | | | | |
| baseline differences | | | | | |
| ATP vs. STP, differences in | | — | — | 0.4 (−0.04 to 0.8) | −0.5 (−3.3 to 2.2) |
| follow-up at 12 months vs. | | | | | |
| baseline differences | | | | | |

^{a}CGI: Severity of Illness.
^{b}Range 1 to 7, higher is more severe.
^{c}GAF: Global Assessment of Functioning.
^{d}Range 0 to 100, higher is better functioning.
^{e}SD: standard deviation.
^{f}From mixed-effects linear regression models adjusted for study site, consulting psychiatrist, and language of the interview, as well as clustering due to patient. The model for global assessment of functioning was further adjusted for clustering due to the referring provider.
^{g}ATP: Asynchronous Telepsychiatry.
^{h}STP: Synchronous Telepsychiatry.

Tables 3 and 4 show descriptive statistics and the results of mixed-effects models for patient self-reported ratings: PHS-12, MHS-12, and PHQ-9, respectively. The pattern of the self-reported ratings was less consistent throughout the follow-up for both ATP and STP arms, with only the mental health score in STP showing statistically significant improvement at 6 months and the PHQ-9 score showing improvement in the ATP group at both 6 and 12 months. However, there were no statistically significant differences in improvement between the arms at any time point for any of the patient self-reported ratings.

TABLE 3
Secondary outcomes: patient self-reported 12-Item Short Form Health Survey
(Physical and Mental) scores at baseline and 6 and 12-month follow-up for the 117
patients included in primary analysis

| | | | | PHS-12 | MHS-12 |
|---|---|---|---|---|---|
| | | PHS-12^{a,b} | MHS-12^{c,d} | Estimate, | Estimate, |
| | n | Mean (SD^{e}) | Mean (SD) | Mean (95% CI)^{f} | Mean (95% CI)^{f} |
| ATP^{g} | | | | | |
| Mean Trajectory | | | | | |
| Baseline | 52 | 39.6 (11.6) | 34.4 (9.6) | — | — |
| Follow-up at 6 months | 51 | 39.5 (11.5) | 36.7 (9.8) | — | — |
| Follow-up at 12 months | 42 | 38.7 (11.5) | 38.2 (9.1) | — | — |
| Change from baseline | | | | | |
| 6 months vs. baseline | 43 | −1.4 (8.8) | 2.0 (11.9) | −1.2 (−3.9 to 1.6) | 2.5 (−0.7 to 5.7) |
| 12 months vs. baseline | 33 | 0.3 (9.3) | 3.7 (12.5) | 0.1 (−3.0 to 3.2) | 3.6 (−0.003 to 7.1) |
| STP^{h} | | | | | |
| Mean Trajectory | | | | | |
| Baseline | 45 | 43.4 (10.4) | 31.7 (8.9) | — | — |
| Follow-up at 6 months | 41 | 41.3 (10.5) | 36.0 (11.1) | — | — |
| Follow-up at 12 months | 28 | 43.9 (9.4) | 34.3 (10.4) | — | — |
| Change from baseline | | | | | |
| 6 months vs. baseline | 34 | −1.8 (11.4) | 5.1 (10.4) | −2.1 (−5.0 to 0.8) | 4.7 (1.4 to 8.1) |
| 12 months vs. baseline | 24 | −1.1 (8.9) | 5.0 (9.9) | 0.001 (−3.3 to 3.3) | 3.7 (−0.2 to 7.5) |
| ATP vs. STP, differences at | | — | — | −9.5 (−32.5 to 13.6) | −2.7 (−24.1 to 18.8) |
| baseline | | | | | |
| ATP vs. STP, differences at | | — | — | −8.6 (−31.5 to 14.4) | −4.9 (−26.1 to 16.3) |
| follow-up at 6 months | | | | | |
| ATP vs. STP, differences at | | — | — | −9.4 (−32.5 to 13.8) | −2.8 (−24.4 to 18.8) |
| follow-up at 12 months | | | | | |
| ATP vs. STP, differences in | | — | — | 0.9 (−3.1 to 4.9) | −2.2 (−6.9 to 2.5) |
| follow-up at 6 months vs. | | | | | |
| baseline differences | | | | | |
| ATP vs. STP, differences in | | — | — | 0.1 (−4.4 to 4.7) | −0.1 (−5.3 to 5.1) |
| follow-up at 12 months vs. | | | | | |
| baseline differences | | | | | |

^{a}PHS-12: 12-Item Short Form Health Survey Physical Health summary score.
^{b}Range 0 to 100, higher is better physical health.
^{c}MHS-12: 12-Item Short Form Health Survey Mental Health summary score.
^{d}Range 0 to 100, higher is better mental health.
^{e}SD: standard deviation.
^{f}From mixed-effects linear regression models adjusted for study site, consulting psychiatrist, and language of the interview, as well as clustering due to patient and primary care provider.
^{g}ATP: Asynchronous Telepsychiatry.
^{h}STP: Synchronous Telepsychiatry.

TABLE 4
Secondary outcomes: patient self-reported Patient Health
Questionnaire-9 scores at baseline and 6 and 12-month follow-up
for the 117 patients included in primary analysis

| | | | PHQ-9^{a} |
|---|---|---|---|
| | | PHQ-9^{a,b} | Estimate, |
| | n | Mean (SD^{c}) | Mean (95% CI)^{d} |
| ATP^{e} | | | |
| Mean Trajectory | | | |
| Baseline | 61 | 12.4 (7.2) | — |
| Follow-up at 6 months | 57 | 9.8 (6.7) | — |
| Follow-up at 12 months | 45 | 10.0 (6.0) | — |
| Change from baseline | | | |
| 6 months vs. baseline | 55 | −2.3 (4.4) | −2.4 (−3.8 to −0.9) |
| 12 months vs. baseline | 43 | −2.8 (5.2) | −2.2 (−3.9 to −0.5) |
| STP^{f} | | | |
| Mean Trajectory | | | |
| Baseline | 53 | 12.6 (6.8) | — |
| Follow-up at 6 months | 40 | 10.8 (6.5) | — |
| Follow-up at 12 months | 34 | 11.9 (7.1) | — |
| Change from baseline | | | |
| 6 months vs. baseline | 40 | −0.7 (4.8) | −0.9 (−2.5 to 0.8) |
| 12 months vs. baseline | 33 | −0.5 (6.4) | −0.7 (−2.4 to 1.0) |
| ATP vs. STP, differences | — | — | 1.8 (−9.4 to 13.1) |
| at baseline | | | |
| ATP vs. STP, differences | — | — | 0.3 (−10.9 to 11.6) |
| at follow-up at 6 months | | | |
| ATP vs. STP, differences | — | — | 0.3 (−11.0 to 11.6) |
| at follow-up at 12 months | | | |
| ATP vs. STP, differences in | — | — | −1.5 (−3.7 to 0.6) |
| follow-up at 6 months vs. | | | |
| baseline differences | | | |
| ATP vs. STP, differences in | — | — | −1.5 (−3.9 to 0.9) |
| follow-up at 12 months vs. | | | |
| baseline differences | | | |

^{a}PHQ-9: Patient Health Questionnaire-9.
^{b}Range 0 to 27, higher is more depressed.
^{c}SD: standard deviation.
^{d}From mixed-effects linear regression models adjusted for study site, consulting psychiatrist, and language of the interview, as well as clustering due to patient and primary care provider.
^{e}ATP: Asynchronous Telepsychiatry.
^{f}STP: Synchronous Telepsychiatry.

The results of the secondary analysis parallel those of the primary analysis, with ATP and STP groups maintaining improvements in both CGI and GAF at 18 and 24 months as compared to baseline and showing no significant interactions between intervention group and follow-up times. Sensitivity analyses adjusted for the baseline score severity confirmed the results of the primary analyses.

At both 12 and 24 months follow up, ATP was not superior to STP in improving patient outcomes. However, both ATP and STP patients had improvements from baseline in clinician-rated outcomes at 12-month (of about 1 point for CGI and 5 points for GAF) and 24-month follow-up (of about 1 point for CGI and 8 points for GAF). The magnitude of these improvements is similar to those found in recent clinical trials on the effect of non-pharmacological interventions on patients' outcomes. A one point improvement in our relatively mildly ill population, as we found, is arguably even more clinically significant than in a population that was more severely ill on average at baseline. Findings of improvement of 8 points on the GAF are similar to findings for long-term therapies in comparable clinical trials.

Patients in both arms had statistically and clinically significant improvements on both clinician-rated outcomes at 6- (estimated difference from baseline for CGI: −0.7, 95% CI −1.0 to 0.4, P<0.001, for ATP and −0.9, 95% CI −1.2 to −0.6, P<0.001, for STP; GAF: 2.7, 95% CI 1.1 to 4.4, P=0.002, for ATP and 3.3, 95% CI 1.4 to 5.1, P<0.001, for STP) and 12-month (estimated difference from baseline: CGI: −0.8, 95% CI −1.1 to −0.5, P<0.001, for ATP and −1.2, 95% CI −1.5 to −0.9, P<0.001, for STP; GAF: 4.7, 95% CI 2.8 to 6.5, P<0.001, for ATP and 5.2, 95% CI 3.2 to 7.2, P<0.001, in STP) follow-up. There were no significant differences in improvement between ATP and STP on any clinician or patient self-reported ratings at any follow-up (all Ps>0.07). Dropout rates were higher than predicted, but similar in the two arms. Of those with baseline visits, 75/160 (47%) did not have a follow-up at 1 year and 107/147 (75%) at 2 years. No serious adverse events were related to the intervention.

### Example 4

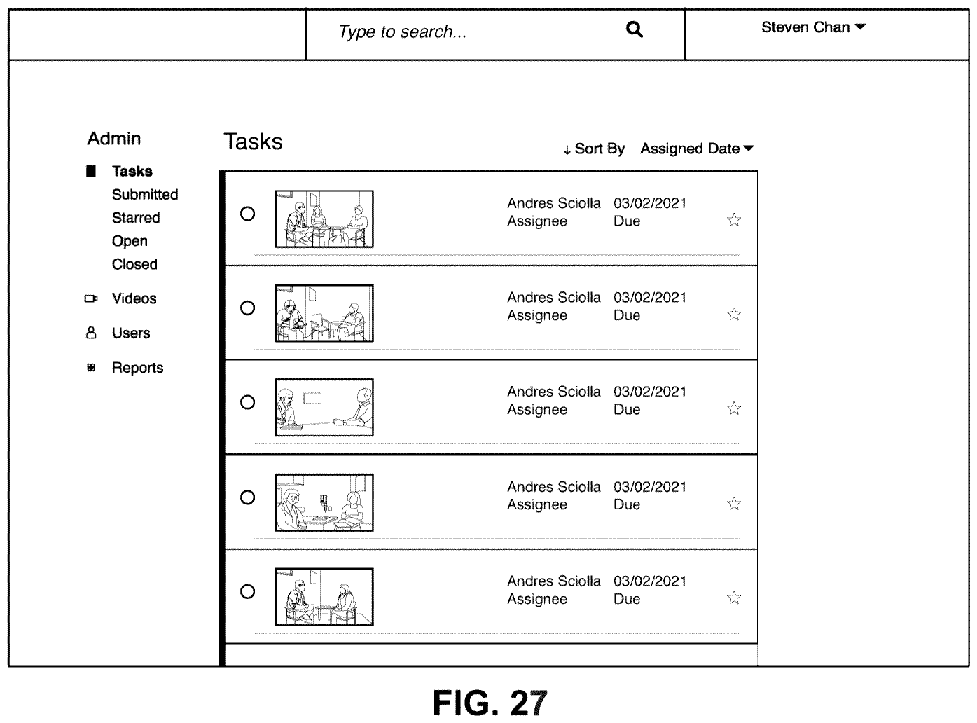

FIGS. 27-32 illustrate graphical user interfaces (GUIs) generated and displayed in a multi-linguistic diagnostic and screening analysis system, according to an embodiment.

At FIG. 27, a healthcare provider may log into a clinic end user device and view a GUI including a list of tasks that various clinicians have been assigned to perform. In some cases, the GUI may indicate the dates the tasks need to be performed by. An administrator of the healthcare provider may view the list of tasks that need to be performed and reorganize them based on various criteria, such as assigned date, importance, open, closed, etc.

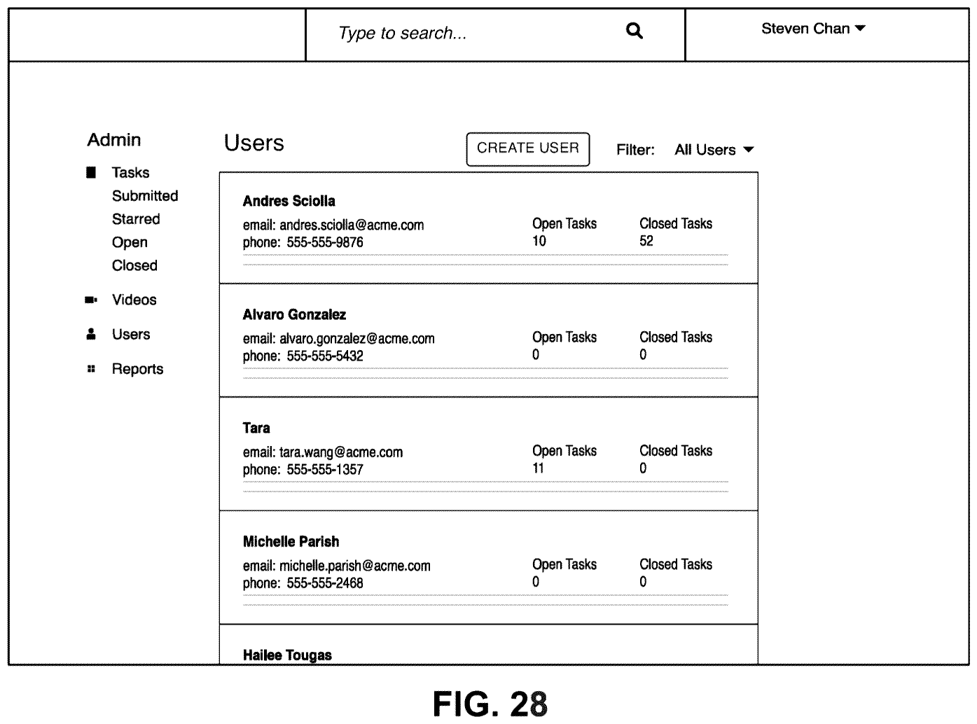

FIG. 28 shows an alternate provider view of a GUI in which an administrator may view aggregated task data for different clinicians. For example, the GUI may illustrate how many tasks individual clinicians have performed and/or how many tasks individual clinicians have finished or are otherwise closed. An administrator may view the list to determine how different clinicians are performing.

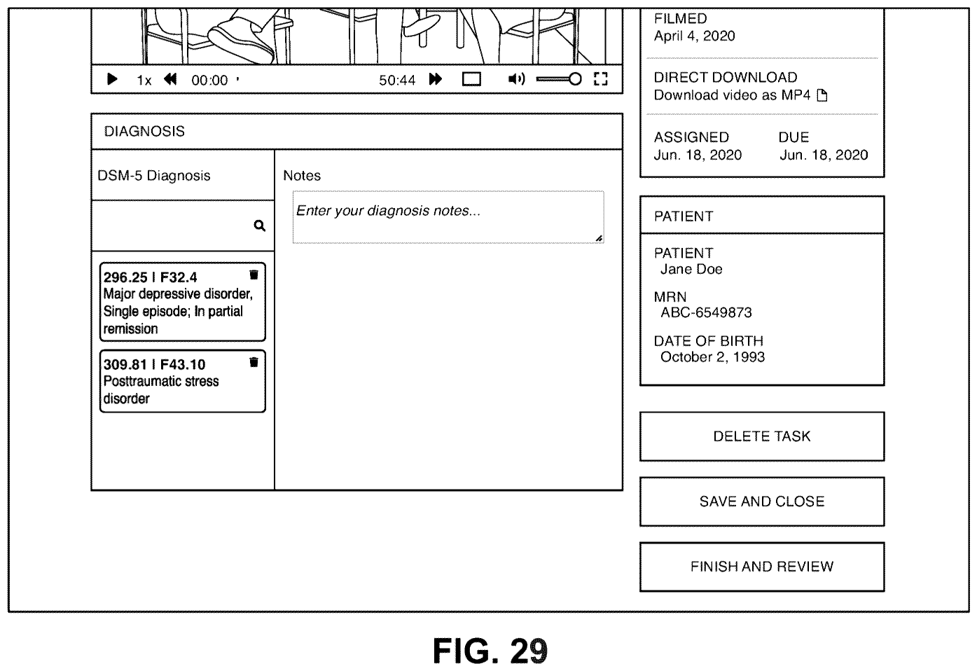

FIG. 29 shows a GUI that a provider may view to view potential diagnoses for a patient. A user may search for diagnoses using the search bar to perform a search of diagnoses with the same or similar letters. A data processing system providing the GUI (e.g., the analytics server 410a) may retrieve diagnoses with matching text and display the retrieved diagnoses on the GUI. The user may select the diagnosis while viewing a video of the clinical encounter or to associate the diagnosis with the patient of the clinical encounter and/or the clinical encounter itself. Accordingly, the user may use the GUI to take live notes of the clinical encounter while viewing a video of the clinical encounter.

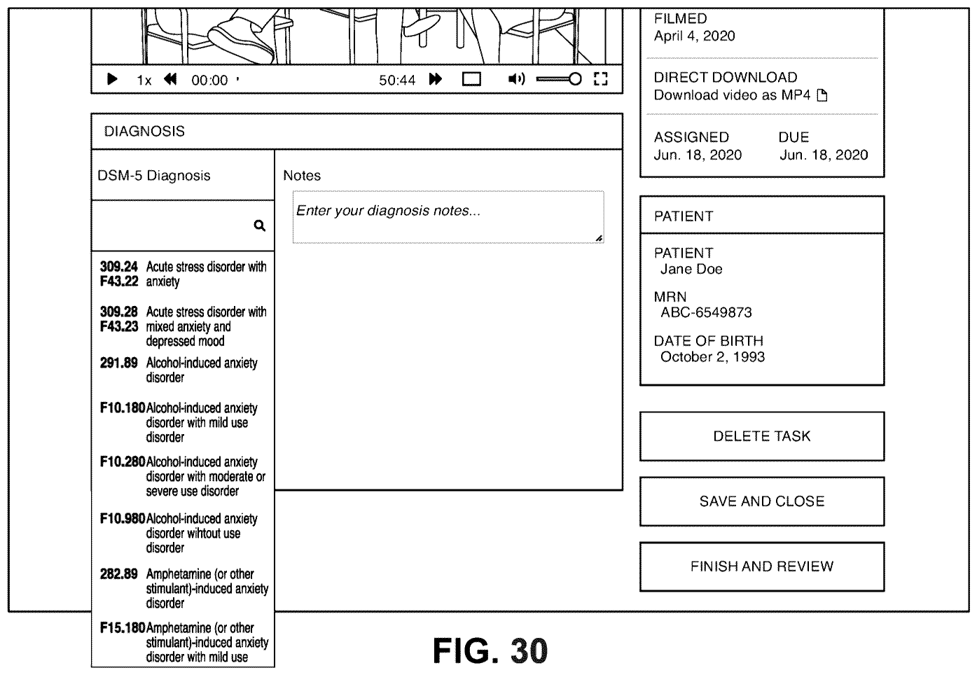

FIG. 30 shows a GUI that a provider may view that indicates selected clinical diagnoses for a patient and/or a clinical encounter. A user may select clinical diagnoses from a dropdown list after performing a search and/or after viewing one or more clinical diagnoses on a user interface selected using machine learning techniques. Upon being selected, the GUI may update to include the selected clinical diagnosis for the patient and/or the clinical encounter.

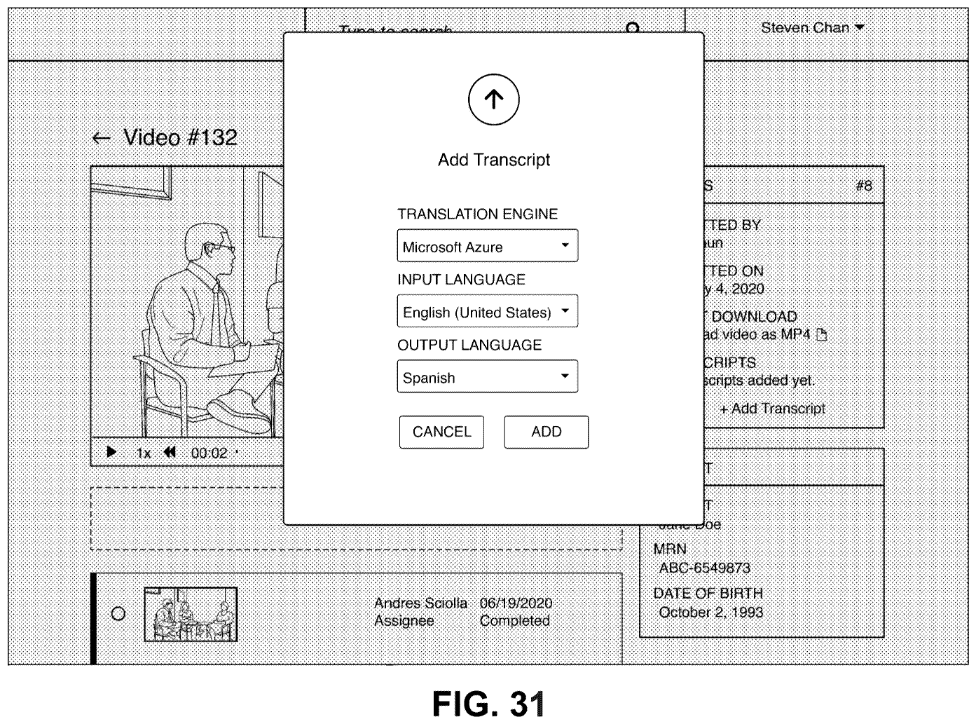

FIG. 31 shows a GUI that a provider may view to obtain a transcript of a clinical encounter in a second language (e.g., a language other than the language being spoken in the video of the clinical encounter). Via the GUI, a user may select a translation engine, an input language of the language spoken during the clinical encounter, and a language into which to translate the language of the clinical encounter. The user may select each option to generate a transcript and/or to cause the words spoken during the clinical encounter to appear in the translated language as text overlaying the video of the clinical encounter.

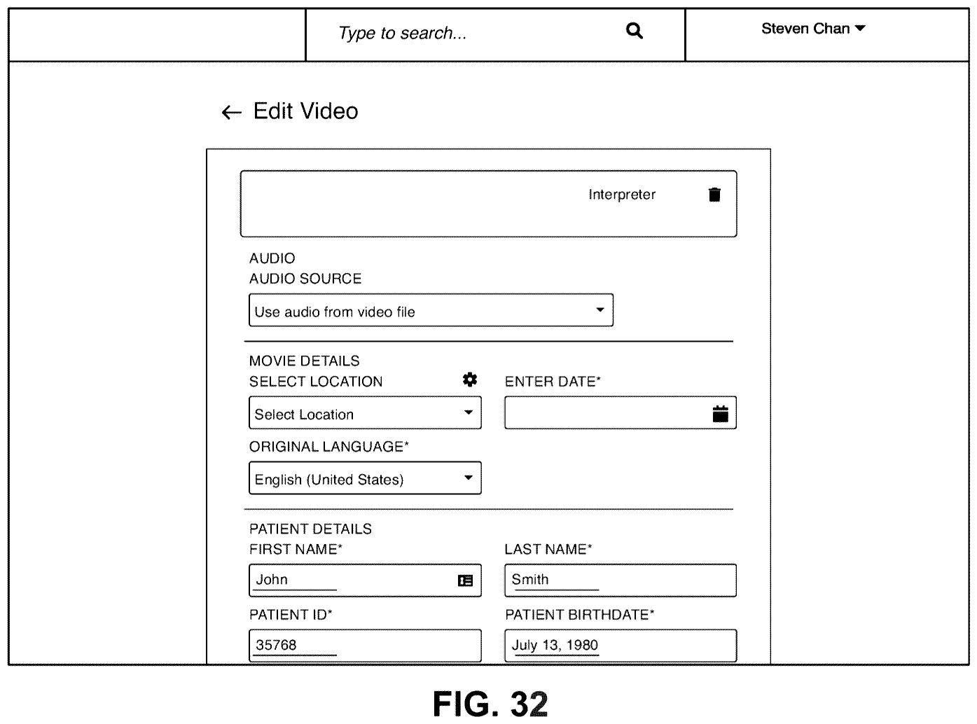

FIG. 32 shows a GUI that a provider may view to configure a user interface for viewing a video of a clinical encounter. Via the GUI, a user may select a source of the audio for a video of the clinical encounter, a location the clinical encounter occurred, the language being spoken in the video of the clinical encounter, and/or patient data of the patient involved in the clinical encounter. In some embodiments, the GUI may also enable the user to select a second language into which to translate the words spoken during the clinical encounter. A data processing system (e.g., the analytics server 410a) may receive such data and use the data to translate the words spoken during the clinical encounter into the second language and/or to predict clinical diagnoses for the patient and/or the clinical encounter. For example, the data processing system may identify a translation service to use based on the two selected languages and use the patient data collected at the user interface in addition to or instead of the words of the clinical encounter to generate the feature vector and predict a clinical diagnosis for the patient. In some cases, the data processing system may retrieve the audio data for processing based on the audio source selected via the GUI (e.g., identify the source and either retrieve the audio data from the source or communicate with a computing device identified by the selected source). In this way, a user can configure and help enable the data processing system to accurately predict a diagnosis for a patient of a clinical encounter.

### Exemplary Embodiments

Exemplary embodiments provided in accordance with the presently disclosed subject matter include, but are not limited to, the claims and the following embodiments:
1. A system for training a model for real-time patient diagnosis, comprising:
   a computer comprising a processor, memory, and a network interface, the processor configured to:
      receive audio data and video data of a clinical encounter, the audio data comprising spoken words by an entity and the video data depicting the entity;
      retrieve clinical data regarding the entity;
      execute a model using the words of the audio data and the retrieved clinical data regarding the entity as input, the execution causing the model to output a plurality of clinical diagnoses for the entity;
      concurrently render the corresponding video data and audio data and the plurality of clinical diagnoses via a computing device associated with a user; and
      store an indication of a selected clinical diagnosis from the plurality of clinical diagnoses responsive to receiving a selection of the clinical diagnosis at the computing device.
2. The system of embodiment 1, wherein the processor is further configured to:
   label a feature vector comprising the words of the audio data and the retrieved clinical data with the indication of the selected clinical diagnosis; and
   train the model with the labeled feature vector.
3. The system of embodiment 1 or 2, wherein the processor is further configured to:
   transcribe the words from the audio data into a text file; and
   convert the words of the audio data from the text file into a second language from a first language,
   wherein concurrently rending the video data and the audio data comprises rendering the words in the second language as text on a display of the computing device.
4. The system of embodiment 3, wherein converting the words of the audio data from the text file into the second language comprises converting the words of the audio data into the second language by executing a first translation service,
   the first translation service selected by the processor from a plurality of translation services by:
      inserting a first text file into each of the plurality of translation services, obtaining a plurality of translated text files each individually associated with a different translation service of the plurality of translation services;
      receiving one or more indications of errors for each of the plurality of translated text files;
      calculating an error rate for each of the plurality of translation services based on the one or more indications of errors; and
      selecting the first translation service responsive to a lowest calculated error rate having an association with the first translation service.
5. The system of any one of embodiments 1 to 4, wherein the processor is further configured to:
   select a clinical treatment plan based on the selected clinical diagnosis; and
   transmit a file comprising the selected clinical treatment plan to the computing device.
6. The system of any one of embodiments 1 to 5, wherein the processor executing the model causes the model to output a confidence score for each of the plurality of clinical diagnoses, the processor further configured to:
   generate a sequential order of the plurality of clinical diagnoses based on the confidence score for each of the plurality of clinical diagnoses, wherein concurrently rendering the plurality of clinical diagnoses comprises rendering text identifying the plurality of clinical diagnoses in the sequential order on a display of the computing device.
7. The system of any one of embodiments 1 to 6, wherein the processor executing the model causes the model to output a confidence score for each of the plurality of clinical diagnoses, and wherein the processor is configured to concurrently render the plurality of clinical diagnoses by rendering the confidence score for each of the plurality of clinical diagnoses on a display of the computing device.
8. The system of any one of embodiments 1 to 7, wherein the processor is further configured to:
   identify one or more characteristics of the patient from the clinical data, the video data, or the audio data; and
   select the model from a plurality of models based on the one or more characteristics.
9. The system of any one of embodiments 1 to 8, wherein the processor is configured to receive the audio data and video data of the clinical encounter by receiving the audio data and video data in real-time during the clinical encounter, and wherein the processor is configured to concurrently render the corresponding video data and audio data and the plurality of clinical diagnoses via the computing device associated with the user by concurrently rendering the corresponding video data and audio data and the plurality of clinical diagnoses in real time during the clinical encounter.
10. The system of embodiment 9, wherein the video data further depicts the user.
11. The system of any one of embodiments 1 to 10, wherein the processor is further configured to:
   extract a term from the audio data comprising spoken words of the entity;
   select a decision tree comprising a set of questions based on the extracted term; and
   sequentially render the set of questions on a display of the computing device during the clinical encounter based on second audio data comprising one or more answers to the set of questions, the answers spoken words by the entity.
12. A method for training a model for real-time patient diagnosis, comprising:
   receiving, by a processor, audio data and video data of a clinical encounter, the audio data comprising spoken words by an entity and the video data depicting the entity;
   retrieving, by the processor, clinical data regarding the entity;
   executing, by the processor, a model using the words of the audio data and the retrieved clinical data regarding the entity as input to output a plurality of clinical diagnoses for the entity;
   concurrently rendering, by the processor, the corresponding video data and audio data and the plurality of clinical diagnoses via a computing device associated with a user; and
   storing, by the processor, an indication of a selected clinical diagnosis from the plurality of clinical diagnoses responsive to receiving a selection of the clinical diagnosis at the computing device.
13. The method of embodiment 12, further comprising:
   labeling, by the processor, a feature vector comprising the words of the audio data and the retrieved clinical data with the indication of the selected clinical diagnosis; and
   training, by the processor, the model with the labeled feature vector.
14. The method of embodiment 12 or 13, further comprising:
   transcribing, by the processor, the words from the audio data into a text file; and
   converting, by the processor, the words of the audio data from the text file into a second language from a first language;
   wherein concurrently rending the video data and the audio data via the computing device comprises rendering, by the processor, the words in the second language as text on a display of the computing device.
15. The method of embodiment 14, wherein converting the words of the audio data from the text file into the second language comprises converting, by the processor, the words of the audio data into the second language by executing, by the processor, a first translation service,
   the first translation service selected by the processor from a plurality of translation services by:
      inserting, by the processor, a first text file into each of the plurality of translation services, obtaining a plurality of translated text files each individually associated with a different translation service of the plurality of translation services;
      receiving, by the processor, one or more indications of errors for each of the plurality of translated text files;
      calculating, by the processor, an error rate for each of the plurality of translation services based on the one or more indications of errors; and
      selecting, by the processor, the first translation service responsive to a lowest calculated error rate having an association with the first translation service.
16. The method of any one of embodiments 12 to 15, further comprising:
   selecting, by the processor, a clinical treatment plan based on the selected clinical diagnosis; and
   transmitting, by the processor, a file comprising the selected clinical treatment plan to the computing device.
17. The method of any one of embodiments 12 to 16, wherein executing the model causes the model to output a confidence score for each of the plurality of clinical diagnoses, and further comprising:
   generating, by the processor, a sequential order of the plurality of clinical diagnoses based on the confidence score for each of the plurality of clinical diagnoses, wherein concurrently rendering the plurality of clinical diagnoses comprises rendering, by the processor, strings identifying the plurality of clinical diagnoses in the sequential order on a display of the computing device.
18. The method of any one of embodiments 12 to 17, wherein executing the model causes the model to output a confidence score for each of the plurality of clinical diagnoses, and wherein concurrently rendering the plurality of clinical diagnoses comprises rendering, by the processor, the confidence score for each of the plurality of clinical diagnoses on a display of the computing device.
19. The method of any one of embodiments 12 to 18, further comprising:
   identifying, by the processor, one or more characteristics of the patient from the clinical data, the video data, or the audio data; and
   selecting, by the processor, the model from a plurality of models based on the one or more characteristics.
20. A non-transitory computer readable medium including encoded instructions that, when executed by a processor of a computer, cause the computer to:
   receive audio data and video data of a clinical encounter, the audio data comprising spoken words by an entity and the video data depicting the entity;
   retrieve clinical data regarding the entity;
   execute a model using the words of the audio data and the retrieved clinical data regarding the entity as input to output a plurality of clinical diagnoses for the entity;
   concurrently render the corresponding video data and audio data and the plurality of clinical diagnoses via a computing device associated with a user; and
   store an indication of a selected clinical diagnosis from the plurality of clinical diagnoses responsive to receiving a selection of the clinical diagnosis at the computing device.

1. A system for training a model for real-time patient diagnosis, comprising:
a computer comprising a processor, memory, and a network interface, the processor configured to:
receive audio data and video data of a clinical encounter, the audio data comprising spoken words by an entity and the video data depicting the entity;
retrieve clinical data regarding the entity;
execute a model using the words of the audio data and the retrieved clinical data regarding the entity as input, the execution causing the model to output a plurality of clinical diagnoses for the entity;
concurrently render the corresponding video data and audio data and the plurality of clinical diagnoses via a computing device associated with a user; and
store an indication of a selected clinical diagnosis from the plurality of clinical diagnoses responsive to receiving a selection of the clinical diagnosis at the computing device.

2. The system of claim 1, wherein the processor is further configured to:
label a feature vector comprising the words of the audio data and the retrieved clinical data with the indication of the selected clinical diagnosis; and
train the model with the labeled feature vector.

3. The system of claim 1, wherein the processor is further configured to:
transcribe the words from the audio data into a text file; and
convert the words of the audio data from the text file into a second language from a first language,
wherein concurrently rending the video data and the audio data comprises rendering the words in the second language as text on a display of the computing device.

4. The system of claim 3, wherein converting the words of the audio data from the text file into the second language comprises converting the words of the audio data into the second language by executing a first translation service,
the first translation service selected by the processor from a plurality of translation services by:
inserting a first text file into each of the plurality of translation services, obtaining a plurality of translated text files each individually associated with a different translation service of the plurality of translation services;
receiving one or more indications of errors for each of the plurality of translated text files;
calculating an error rate for each of the plurality of translation services based on the one or more indications of errors; and
selecting the first translation service responsive to a lowest calculated error rate having an association with the first translation service.

5. The system of claim 1, wherein the processor is further configured to:
select a clinical treatment plan based on the selected clinical diagnosis; and
transmit a file comprising the selected clinical treatment plan to the computing device.

6. The system of claim 1, wherein the processor executing the model causes the model to output a confidence score for each of the plurality of clinical diagnoses, the processor further configured to:
generate a sequential order of the plurality of clinical diagnoses based on the confidence score for each of the plurality of clinical diagnoses, wherein concurrently rendering the plurality of clinical diagnoses comprises rendering text identifying the plurality of clinical diagnoses in the sequential order on a display of the computing device.

7. The system of claim 1, wherein the processor executing the model causes the model to output a confidence score for each of the plurality of clinical diagnoses, and wherein the processor is configured to concurrently render the plurality of clinical diagnoses by rendering the confidence score for each of the plurality of clinical diagnoses on a display of the computing device.

8. The system of claim 1, wherein the processor is further configured to:
identify one or more characteristics of the patient from the clinical data, the video data, or the audio data; and
select the model from a plurality of models based on the one or more characteristics.

9. The system of a 1, wherein the processor is configured to receive the audio data and video data of the clinical encounter by receiving the audio data and video data in real-time during the clinical encounter, and wherein the processor is configured to concurrently render the corresponding video data and audio data and the plurality of clinical diagnoses via the computing device associated with the user by concurrently rendering the corresponding video data and audio data and the plurality of clinical diagnoses in real time during the clinical encounter.

10. The system of claim 9, wherein the video data further depicts the user.

11. The system of claim 1, wherein the processor is further configured to:
extract a term from the audio data comprising spoken words of the entity;
select a decision tree comprising a set of questions based on the extracted term; and
sequentially render the set of questions on a display of the computing device during the clinical encounter based on second audio data comprising one or more answers to the set of questions, the answers spoken words by the entity.

12. A method for training a model for real-time patient diagnosis, comprising:
receiving, by a processor, audio data and video data of a clinical encounter, the audio data comprising spoken words by an entity and the video data depicting the entity;
retrieving, by the processor, clinical data regarding the entity;
executing, by the processor, a model using the words of the audio data and the retrieved clinical data regarding the entity as input to output a plurality of clinical diagnoses for the entity;
concurrently rendering, by the processor, the corresponding video data and audio data and the plurality of clinical diagnoses via a computing device associated with a user; and
storing, by the processor, an indication of a selected clinical diagnosis from the plurality of clinical diagnoses responsive to receiving a selection of the clinical diagnosis at the computing device.

13. The method of claim 12, further comprising:
labeling, by the processor, a feature vector comprising the words of the audio data and the retrieved clinical data with the indication of the selected clinical diagnosis; and
training, by the processor, the model with the labeled feature vector.

14. The method of claim 12, further comprising:
transcribing, by the processor, the words from the audio data into a text file; and
converting, by the processor, the words of the audio data from the text file into a second language from a first language;
wherein concurrently rending the video data and the audio data via the computing device comprises rendering, by the processor, the words in the second language as text on a display of the computing device.

15. The method of claim 14, wherein converting the words of the audio data from the text file into the second language comprises converting, by the processor, the words of the audio data into the second language by executing, by the processor, a first translation service,
the first translation service selected by the processor from a plurality of translation services by:
inserting, by the processor, a first text file into each of the plurality of translation services, obtaining a plurality of translated text files each individually associated with a different translation service of the plurality of translation services;
receiving, by the processor, one or more indications of errors for each of the plurality of translated text files;
calculating, by the processor, an error rate for each of the plurality of translation services based on the one or more indications of errors; and
selecting, by the processor, the first translation service responsive to a lowest calculated error rate having an association with the first translation service.

16. The method of claim 12, further comprising:
selecting, by the processor, a clinical treatment plan based on the selected clinical diagnosis; and
transmitting, by the processor, a file comprising the selected clinical treatment plan to the computing device.

17. The method of 12, wherein executing the model causes the model to output a confidence score for each of the plurality of clinical diagnoses, and further comprising:
generating, by the processor, a sequential order of the plurality of clinical diagnoses based on the confidence score for each of the plurality of clinical diagnoses, wherein concurrently rendering the plurality of clinical diagnoses comprises rendering, by the processor, strings identifying the plurality of clinical diagnoses in the sequential order on a display of the computing device.

18. The method of m 12, wherein executing the model causes the model to output a confidence score for each of the plurality of clinical diagnoses, and wherein concurrently rendering the plurality of clinical diagnoses comprises rendering, by the processor, the confidence score for each of the plurality of clinical diagnoses on a display of the computing device.

19. The method of claim 12, further comprising:
identifying, by the processor, one or more characteristics of the patient from the clinical data, the video data, or the audio data; and
selecting, by the processor, the model from a plurality of models based on the one or more characteristics.

20. A non-transitory computer readable medium including encoded instructions that, when executed by a processor of a computer, cause the computer to:
receive audio data and video data of a clinical encounter, the audio data comprising spoken words by an entity and the video data depicting the entity;
retrieve clinical data regarding the entity;
execute a model using the words of the audio data and the retrieved clinical data regarding the entity as input to output a plurality of clinical diagnoses for the entity;
concurrently render the corresponding video data and audio data and the plurality of clinical diagnoses via a computing device associated with a user; and store an indication of a selected clinical diagnosis from the plurality of clinical diagnoses responsive to receiving a selection of the clinical diagnosis at the computing device.
